(19) United States
(12) Patent Application Publication
(10) Pub. No.: US 2020/0016256 A1
(43) Pub. Date: Jan. 16, 2020

(54) IMMUNE RESPONSE MODULATION USING LIVE BIOTHERAPEUTICS, FOR CONDITIONS SUCH AS ALLERGY DESENSITIZATION

(71) Applicants: THE UNIVERSITY OF CHICAGO, Chicago, IL (US); GUSTO GLOBAL, LLC, Charlotte, NC (US)

(72) Inventors: Jack A. GILBERT, Naperville, IL (US); Daniel van der LELIE, Charlotte, NC (US); Safiyh TAGHAVI, Charlotte, NC (US)

(73) Assignees: The University of Chicago, Chicago, IL (US); Gusto Global, LLC, Charlotte, NC (US)

(21) Appl. No.: 16/335,636

(22) PCT Filed: Sep. 22, 2017
(86) PCT No.: PCT/US2017/052988

§ 371 Date: Mar. 21, 2019

Related U.S. Application Data
(60) Provisional application No. 62/398,041, filed on Sep. 22, 2016.

(51) Int. Cl. A61K 39/02; A61K 39/05; G01N 33/569; A61P 37/08; A61K 35/742; A23L 33/135

(52) U.S. Cl. CPC A61K 39/0216; A61K 39/05; G01N 33/56911; A61K 2039/55; A61K 35/742; A23L 33/135; A61P 37/08

(57) ABSTRACT

Provided herein are compositions (e.g., probiotic, pharmaceutical, etc.) comprising one or more strains of non-Clostridia class bacteria and methods of use thereof for allergen desensitization. In particular, bacteria of bacterial classes such as Negativicutes, Actinobacteria, and Bacteroidia support allergen desensitization, for example, by promoting production of metabolites that aid in desensitization or performing catabolism of food allergens.

### FIELD

Provided herein are compositions (e.g., probiotic, therapeutics, pharmaceutical, etc.) comprising one or more strains of non-Clostridium clusters IV and XIVa bacteria (e.g., non-Clostridia class bacteria) and methods of use thereof for immune modulation, resulting in e.g. decreased inflammatory responses and/or allergen desensitization. In particular, bacteria of bacterial classes such as Negativicutes, Actinobacteria, and Bacteroidia support allergen desensitization, for example, by promoting production of metabolites that help to modulate the immune response resulting in desensitization. This effect is further enhanced by performing catabolism of allergens.

### BACKGROUND

Over activity of the immune system can result in many conditions, such as asthma, allergic rhinitis, eczema, IBD, IBS Ulcerative Colitis and Crohn's disease. It can also result in various forms of food allergies, such as allergies to peanuts, shellfish, and dairy products. With an estimated prevalence of 2-3% worldwide, Cow's milk allergy (CMA) is one of the most important food allergies of the early childhood. In addition, a strong correlation has been reported between early childhood CMA and the onset of other allergies at later age. The gastrointestinal (GI) microbiota play a crucial role in the acquisition of tolerance in allergic (CMA) infants (Berni Canani et al., 2015; incorporated by reference in its entirety). A recent study showed that dietary management with a formula containing an Extensively Hydrolyzed Casein Formulation (EHCF), supplemented with the probiotic strain of Lactobacillus rhamnosus GG (LGG), resulted in a higher rate of cow's milk tolerance in sensitive infants compared to infants treated with the EHCF without a probiotic (Berni Canani et al., 2015; incorporated by reference in its entirety). Amplicon sequencing analysis using 16S rRNA revealed that L. rhamnosus GG supplementation caused the enrichment of specific strains of potential butyrate producing bacterial genera, Rosebuira, Coprococcus and Ruminococcus (all Class XIVa Clostridia). The majority of probiotic formulations that are being explored for modulating the immune response and de-sensitization of animal models or human subjects to food allergens revolve around the addition of human-derived butyrate-producing bacterial species that belong to the Clostridia classes IV and XVIa to induce the accumulation of regulatory T cells that lead to the control of inflammation, a decrease in the secretion of a pro-inflammatory cytokine, or an enhanced secretion of an anti-inflammatory cytokine by a population of human peripheral blood mononuclear cells. Such probiotic formulations have proven useful in the treatment of e.g. allergies and other immune-disorders in only a subset of patients. What is needed is alternative desensitization formulations or probiotic formulations to supplement those already in use, to provide immune modulation and enhanced desensitization to a broader group of patients.

### SUMMARY

Provided herein are compositions (e.g., probiotic, therapeutics, pharmaceutical, etc.) comprising one or more strains of non-Clostridium clusters IV and XIVa bacteria (e.g., non-Clostridia class bacteria (e.g., Acidaminococcus intestini, Alistipes putredinis, Bacteroides massiliensis, Bacteroides stercoris, Bifidobacterium adolescentis, Megamonas funiformis, Megamonas hypermegale, Megamonas rupellensis, and taxonomically-related bacteria that similarly support allergen tolerance), Clostridia of clusters other than clusters IV and XIVa, etc.) and methods of use thereof for immune modulation, e.g. controlling inflammation, allergen desensitization and treatment of allergic response. In particular, bacteria of bacterial classes such as Actinobacteria, Negativicutes and Bacteroidia allow for immune modulation and support allergen desensitization, for example, by promoting production of metabolites that modify the immune response, aid in desensitization or performing catabolism of allergens. Bacteria may be administered in any suitable state, for example, live (e.g., vegetative), freeze-dried, as spores, etc. Accordingly, provided herein are technologies related to a method of immune response modulation, allowing for treating/preventing immune response dependent conditions, such as chronic inflammations and allergies (e.g., preventing development of sensitivity to an allergen, desensitizing a subject to an allergen, etc.) in a subject. In some embodiments, the technology provides a method comprising administering a composition comprising non-Clostridium clusters IV and XIVa bacteria bacteria of a non-Clostridia class to the subject (e.g., co-administered with one or more non-Clostridium clusters IV and XIVa bacteria, administered without Clostridia class bacteria, etc.). In some embodiments, methods comprise administering a composition comprising at least 10⁴ colony forming units (CFU) (e.g., at least 1×10⁴ CFU, 2×10⁴ CFU, 5×10⁴ CFU, 1×10⁵ CFU, 2×10⁵ CFU, 5×10⁵ CFU, 1×10⁶ CFU, 2×10⁶ CFU, 5×10⁶ CFU, 1×10⁷ CFU, 2×10⁷ CFU, 5×10⁷ CFU, 1×10⁸ CFU, 2×10⁸ CFU, 5×10⁸ CFU, 1×10⁹ CFU, 2×10⁹ CFU, 5×10⁹ CFU, 1×10¹⁰ CFU, 2×10¹⁰ CFU, 5×10¹⁰ CFU, 1×10¹¹ CFU, 2×10¹¹ CFU, 5×10¹¹ CFU, 1×10¹² CFU, 2×10¹² CFU, 5×10¹² CFU, or more or ranges there between) of one or more non-Clostridia class bacteria. In some embodiments, methods comprise administering a composition comprising bacterial spores.

In some embodiments, provided herein are pharmaceutical compositions comprising non-Clostridium clusters IV and XIVa bacteria (e.g., non-Clostridia class bacteria, Clostridia of clusters other than clusters IV and XIVa, etc.) and a pharmaceutically acceptable carrier. In some embodiments, the non-Clostridium clusters IV and XIVa bacteria are non-Clostridia class bacteria. In some embodiments, the non-Clostridia class bacteria comprises one or more species selected from the phyla Actinobacteria, Bacteroidetes, and Firmicutes. In some embodiments, the non-Clostridia class bacteria comprises one or more species selected from the phylum Actinobacteria and genus Bifidobacteria. In some embodiments, the non-Clostridia class bacteria comprises Bifidobacterium adolescentis. In some embodiments, the non-Clostridia class bacteria comprises one or more species selected from the phylum Bacteroidetes and the class Bacteroidia. In some embodiments, the non-Clostridia class bacteria comprises one or more species of a genus selected from the group consisting of Rikenella, Alistipes, Anaerocella, Porphyromonas, Prevotella, Hallella, and Alloprevotella. In some embodiments, the non-Clostridia class bacteria comprises a Bacteroidia species selected from the group consisting of Alistipes putredinis, Bacteroides massiliensis, and Bacteroides stercoris. In some embodiments, the non-Clostridia class bacteria comprises one or more species selected from the phylum Firmicutes and the class Negativicutes. In some embodiments, the non-Clostridia class bacteria comprises one or more species of a genus selected from the group consisting of Megamonas, Acidaminococcus, Succinispira, Megasphaera, Dialister, Pelosiunus, and Veillonella. In some embodiments, the non-Clostridia class bacteria comprises one or more species selected from the group consisting of Acidaminococcus intestini, Megamonas funiformis, Megamonas hypermegale, and Megamonas rupellensis. In some embodiments, the non-Clostridia class bacteria comprises bacteria of one or more genera selected from the group consisting of Megamonas, Acidaminococcus, Succinispira, Megasphaera, Dialister, Pelosiunus, Veillonella, Rikenella, Alistipes, Anaerocella, Porphyromonas, Prevotella, Hallella, and Alloprevotella. In some embodiments, the non-Clostridia class bacteria comprises one or more species selected from the group consisting of Acidaminococcus intestini, Alistipes putredinis, Bacteroides massiliensis, Bacteroides stercoris, Bifidobacterium adolescentis, Megamonas funiformis, Megamonas hypermegale, Megamonas rupellensis, and taxonomically-related bacteria that similarly support allergen tolerance. In some embodiments, the non-Clostridium clusters IV and XIVa bacteria are Clostridia of clusters other than clusters IV and XIVa. In some embodiments, the pharmaceutical composition comprises a therapeutically effective amount of non-Clostridium clusters IV and XIVa bacteria. In some embodiments, a therapeutically effective amount of non-Clostridium clusters IV and XIVa bacteria (e.g., non-Clostridia class bacteria) is an amount sufficient to increase butyrate production by Clostridia class bacteria, including Clostridia classes IV and XIVa bacteria, in the subject. In some embodiments, a therapeutically effective amount of non-Clostridium clusters IV and XIVa bacteria (e.g., non-Clostridia class bacteria) is an amount sufficient to activate regulator T cell accumulation in the subject, to cause a decrease in the secretion of a pro-inflammatory cytokine or an enhanced secretion of an anti-inflammatory cytokine by a population of human peripheral blood mononuclear cells at levels sufficient to allow for immune response modulation. In some embodiments, a therapeutically effective amount of non-Clostridium clusters IV and XIVa bacteria (e.g., non-Clostridia class bacteria) is an amount sufficient to increase catabolism of allergens in the subject. In some embodiments, the composition comprises at least 10⁴ colony forming units (CFU) of non-Clostridium clusters IV and XIVa bacteria (e.g., non-Clostridia class bacteria). In some embodiments, the pharmaceutical composition further comprises a probiotic or a prebiotic. In some embodiments, the pharmaceutical composition is formulated for administration to a human newborn, neonate, infant, or child. In some embodiments, the bacteria are alive. In some embodiments, the bacteria are in a vegetative stage or sporulated. In some embodiments, the pharmaceutical composition is formulated for oral administration. In some embodiments, the pharmaceutical composition is formulated for rectal administration. In some embodiments, the pharmaceutical composition is a nutraceutical or a food.

Methods are provided for the treatment of subjects in need of treatment for immune response modulation (e.g., subjects with inflammatory conditions and/or immune hypersensitivity to a particular allergen or set of allergens (e.g., including milk or milk proteins)) with non-Clostridium clusters IV and XIVa bacteria (e.g., non-Clostridia class bacteria (e.g., Acidaminococcus intestini, Alistipes putredinis, Bacteroides massiliensis, Bacteroides stercoris, Bifidobacterium adolescentis, Megamonas funiformis, Megamonas hypermegale, Megamonas rupellensis, and taxonomically-related bacteria that similarly support allergen tolerance)). In other embodiments, methods are provided for the prevention of development of inflammatory responses and/or allergen hypersensitivity in a subject (e.g., a subject at increased risk of allergy development). In some embodiments, bacterial compositions described herein (e.g., comprising non-Clostridia class bacteria) are administered to a subject having gut microbiota that places the subject at risk of developing inflammatory responses and/or allergen hypersensitivity. In some embodiments, bacterial compositions described herein (e.g., comprising non-Clostridium clusters IV and XIVa bacteria (e.g., non-Clostridia class bacteria)) are administered to a subject having gut microbiota that has caused the subject to experience inflammatory conditions and/or allergen hypersensitivity. In some exemplary embodiments, methods comprise treating a subject who has a gut microbiota that differs from the normal microbiota in one or both of membership or relative abundance of one or more members of the gut microbiota, e.g., methods comprise administering a composition comprising non-Clostridium clusters IV and XIVa bacteria (e.g., non-Clostridia class bacteria) to a subject who has a gut microbiota that differs from the microbiota, in one or both of membership or relative abundance of one or more members of gut microbiota that are preventative of inflammatory responses and/or hypersensitivity to allergens. In some embodiments, the technology relates to methods comprising treating a subject that has a gut microbiota that differs from the normal microbiota (e.g., microbiota that promotes allergen tolerance) in the membership or relative abundance of non-Clostridium clusters IV and XIVa bacteria (e.g., non-Clostridia class bacteria), e.g., methods comprising administering a composition comprising non-Clostridium clusters IV and XIVa bacteria (e.g., non-Clostridia class bacteria) to a subject who has a gut microbiota that differs from the normal microbiota (e.g., microbiota that promotes allergen tolerance) in the membership or relative abundance of the beneficial non-Clostridium clusters IV and XIVa bacteria (e.g., non-Clostridia class bacteria).

In some embodiments, provided herein are methods of preventing inflammatory responses e.g. caused by allergen hypersensitivity in a subject, the methods comprising administering a composition comprising non-Clostridium clusters IV and XIVa bacteria (e.g., non-Clostridia class bacteria) to the subject. In some embodiments, the subject is at risk of developing inflammatory conditions, e.g. caused by allergen hypersensitivity (e.g., increased risk based on family history of asthma or allergies, genetic factors (e.g., determined from genetic testing), abnormal gut microbiota, age (children are more likely to develop an allergy than are adults), suffering from asthma or another allergy, etc.). In some embodiments, provided herein are methods of treating a subject suffering from inflammatory conditions such as allergies (e.g., food allergies, environmental allergies (e.g., pollen, dust mites, pet dander, mold, mildew, etc.), seasonal allergies, etc.) or other atopy diseases (e.g., allergic rhinitis, eczema, etc.), the methods comprising administering a composition comprising non-Clostridium clusters IV and XIVa bacteria (e.g., non-Clostridia class bacteria) to the subject. In some embodiments, provided herein are methods of desensitizing a subject to an allergen, the methods comprising administering a composition comprising non-Clostridium clusters IV and XIVa bacteria (e.g., non-Clostridia class bacteria) to the subject. In some embodiments, the subject suffers from hypersensitivity to an allergen. In some embodiments, the subject suffers from hypersensitivity to one or more allergens. In some embodiments, the subject suffers from hypersensitivity to one or more foods (e.g., food allergens) from the group consisting of milk, milk proteins, eggs, fish, hazelnuts, walnuts, almonds, Brazil nuts, peanuts, shrimps, mussels, crab, soy, and wheat. In some embodiments, the subject suffers atopic syndrome. In some embodiments, the subject has abnormal gut microbiota. In some embodiments, the subject is a human. In some embodiments, the subject is a human infant, neonate, or child. In some embodiments, the non-Clostridium clusters IV and XIVa bacteria (e.g., non-Clostridia class bacteria) comprises one or more species selected from the phyla Actinobacteria, Bacteroidetes, and Firmicutes. In some embodiments, the non-Clostridium clusters IV and XIVa bacteria (e.g., non-Clostridia class bacteria) comprises one or more species selected from the phylum Actinobacteria and genus Bifidobacteria. In some embodiments, the non-Clostridium clusters IV and XIVa bacteria (e.g., non-Clostridia class bacteria) comprises Bifidobacterium adolescentis. In some embodiments, the non-Clostridium clusters IV and XIVa bacteria (e.g., non-Clostridia class bacteria) comprises one or more species selected from the phylum Bacteroidetes and the class Bacteroidia. In some embodiments, the non-Clostridium clusters IV and XIVa bacteria (e.g., non-Clostridia class bacteria) comprises one or more species of a genus selected from the group consisting of Rikenella, Alistipes, Anaerocella, Porphyromonas, Prevotella, Hallella, and Alloprevotella. In some embodiments, the non-Clostridium clusters IV and XIVa bacteria (e.g., non-Clostridia class bacteria) comprises a Bacteroidia species selected from the group consisting of Alistipes putredinis, Bacteroides massiliensis, and Bacteroides stercoris. In some embodiments, the non-Clostridium clusters IV and XIVa bacteria (e.g., non-Clostridia class bacteria) comprises one or more species selected from the phylum Firmicutes and the class Negativicutes. In some embodiments, the non-Clostridium clusters IV and XIVa bacteria (e.g., non-Clostridia class bacteria) comprises one or more species of a genus selected from the group consisting of Megamonas, Acidaminococcus, Succinispira, Megasphaera, Dialister, Pelosiunus, and Veillonella. In some embodiments, the non-Clostridium clusters IV and XIVa bacteria (e.g., non-Clostridia class bacteria) comprises one or more species selected from the group consisting of Acidaminococcus intestini, Megamonas funiformis, Megamonas hypermegale, and Megamonas rupellensis. In some embodiments, the non-Clostridium clusters IV and XIVa bacteria (e.g., non-Clostridia class bacteria) comprises bacteria of one or more genera selected from the group consisting of Megamonas, Acidaminococcus, Succinispira, Megasphaera, Dialister, Pelosiunus, Veillonella, Rikenella, Alistipes, Anaerocella, Porphyromonas, Prevotella, Hallella, and Alloprevotella. In some embodiments, the non-Clostridium clusters IV and XIVa bacteria (e.g., non-Clostridia class bacteria) comprises one or more species selected from the group consisting of Acidaminococcus intestini, Alistipes putredinis, Bacteroides massiliensis, Bacteroides stercoris, Bifidobacterium adolescentis, Megamonas funiformis, Megamonas hypermegale, Megamonas rupellensis, and taxonomically-related bacteria that similarly support immune modulation resulting in e.g. allergen tolerance or decreased inflammation. non-Clostridium clusters IV and XIVa bacteria comprises Claostridia class bacteria of clusters other than IV and XIVa. In some embodiments, administering the composition supports butyrate production by Clostridia class bacteria in the subject. In some embodiments, administering the composition activates regulator T cell accumulation, and can cause a decrease in the secretion of a pro-inflammatory cytokine or an enhanced secretion of an anti-inflammatory cytokine by a population of human peripheral blood mononuclear cells at levels sufficient to allow for immune response modulation. In some embodiments, administering the composition results in increased catabolism of allergens. In some embodiments, the composition comprises at least 10⁴ colony forming units (CFU) of non-Clostridium clusters IV and XIVa bacteria (e.g., non-Clostridia class bacteria). In some embodiments, the composition is administered orally. In some embodiments, the composition is administered rectally. In some embodiments, treatment further comprises assaying the microbiome and/or metabolome of the subject. In some embodiments, assaying the microbiome comprises testing the presence, absence, or amount of one or more non-Clostridium clusters IV and XIVa bacteria (e.g., non-Clostridia class bacteria) and/or Clostridia bacteria in the gut of the subject. In some embodiments, assaying the metabolome comprises quantifying amount of one or more metabolites in the gut of the subject. In some embodiments, one of said one or more metabolites is butyrate. In some embodiments, the assaying is performed on the subject before and/or after administration of the composition. In some embodiments, the composition is co-administered with one or more additional active agents. In some embodiments, the additional active agent comprises a probiotic component or a prebiotic component. In some embodiments, the additional active agent comprises Clostridia class bacteria, including Clostridia class IV and/or XIVa bacteria.

The technology is not limited in the types or classes of subjects or patients that are treated and/or that are administered with the compositions comprising non-Clostridium clusters IV and XIVa bacteria (e.g., non-Clostridia class bacteria (e.g., Acidaminococcus intestini, Alistipes putredinis, Bacteroides massiliensis, Bacteroides stercoris, Bifidobacterium adolescentis, Megamonas funiformis, Megamonas hypermegale, Megamonas rupellensis, and taxonomically-related bacteria that similarly support allergen tolerance)). For example, in some embodiments the subject is a human. In some embodiments, the subject is a young human, e.g., that has not developed a gut microbiota that helps to train the immune system, helps to control inflammatory conditions, and/or promotes allergen tolerance. For example, in some embodiments the subject is a human infant or a human neonate or a human newborn. The technology is applicable to subjects and patients that are nonhuman, e.g., mammals, birds, etc., including but not limited to livestock animals, domesticated animals, animals in captivity, etc. In some embodiments, the subject is a human that has an age of 1 to 60 minutes (e.g., 1, 2, 3, 5, 10, 15, 20, 25, 30, 35, 40, 45, 50, 55, 60 minutes old, e.g., 1, 2, 3, 5, 10, 15, 20, 25, 30, 35, 40, 45, 50, 55, 60 minutes after birth); in some embodiments, the subject is a human that has an age of from 1 to 24 hours (e.g., 1, 2, 3, 4, 5, 6, 7, 8, 9, 10, 12, 13, 14, 15, 16, 17, 18, 19, 20, 21, 22, 23, 24 hours, e.g., 1, 2, 3, 4, 5, 6, 7, 8, 9, 10, 12, 13, 14, 15, 16, 17, 18, 19, 20, 21, 22, 23, 24 hours after birth); in some embodiments the subject is a human that has an age of 1 day, 2, days, 3, days, 4 days, 5 days, 6 days, 1 week, 2 weeks, 3 weeks, one month, 2 months, 4 months, 6 months, 9 months, 1 year, 2 year, 4 years, 6 years, 8 years, 10 years, 12 years, 14 years, 16 years, 18 years, 20 years, 30 years, 40 years, 50 years, 60 years, or older, or any ranges there between.

In some embodiments, the subject is a juvenile, adult, or elderly subject. In some embodiments, the subject has recently (e.g., within 1 week, 2 weeks, 1 month 2 months, 6 months, 1 year, 2 years, or more or ranges there between) developed or become symptomatic of a (chronic) inflammatory condition, e.g. hypersensitivity to an allergen (e.g., milk or milk protein). In some embodiments, the subject has an abnormal or pathogenic gut microbiota (e.g., gut microbiota that promote and/or are permissive of development and/or maintenance of inflammatory conditions and/or allergen hypersensitivity).

The technology is not limited in the type or route if administration. In some embodiments, the type or route of administration provides the composition comprising non-Clostridium clusters IV and XIVa bacteria (e.g., non-Clostridia class bacteria) to the subject's gastrointestinal tract. For example, in some embodiments the composition is administered orally and in some embodiments the composition is administered rectally.

Some embodiments comprise administering compositions comprising non-Clostridium clusters IV and XIVa bacteria (e.g., non-Clostridia class bacteria (e.g., Acidaminococcus intestini, Alistipes putredinis, Bacteroides massiliensis, Bacteroides stercoris, Bifidobacterium adolescentis, Megamonas funiformis, Megamonas hypermegale, Megamonas rupellensis, and taxonomically-related bacteria that similarly support allergen tolerance)) and one or more additional components. In some embodiments, additional components are selected from Clostridia class bacteria (e.g., Blautia hydrogenotrophica, Marvinbryantia formatexigens, Ruminococcus gnavis, and taxonomically-related bacteria that similarly support allergen tolerance) and non-bacterial components (e.g., to assist in allergen desensitization, for formulation of the composition (e.g., stability, shelf-life, consistency, taste, strain engrafting, strain activity, etc.), etc.).

In some embodiments, the technology comprises testing a subject or a patient. For example, some embodiments comprise testing the subject for the presence, absence, or amount of non-Clostridium clusters IV and XIVa bacteria (e.g., non-Clostridia class bacteria (e.g., specific taxa of non-Clostridia bacteria)) and/or Clostridia class bacteria in the gut microbiota. In some embodiments, embodiments, a subject is tested for the presence of metabolites that promote hypersensitivity to allergens, promote allergen tolerance, etc. Some embodiments comprise testing the subject for an allergic response to one or more allergens (e.g., milk or milk protein). Some embodiments comprise testing the subject for an abnormal gut microbiota (e.g., microbiota that promotes development or maintenance of allergen hypersensitivity). The technology provides methods in which a subject or a patient is tested before and/or after administration of a composition comprising non-Clostridium clusters IV and XIVa bacteria (e.g., non-Clostridia class bacteria) to the subject or patient. In some embodiments, the testing informs the dose amount, dose schedule, and/or CFU of non-Clostridium clusters IV and XIVa bacteria (e.g., non-Clostridia class bacteria) in the composition that is administered to the subject or patient. Some embodiments comprise administration of a composition comprising non-Clostridium clusters IV and XIVa bacteria (e.g., non-Clostridia class bacteria) to the subject or patient, testing the subject or patient, and a second administration of a composition comprising non-Clostridium clusters IV and XIVa bacteria (e.g., non-Clostridia class bacteria) to the subject. The first and second administrations and/or compositions may be the same or different, e.g., same or different in dose, amount, route, composition, species of non-Clostridium clusters IV and XIVa bacteria (e.g., non-Clostridia class bacteria), CFU of non-Clostridium clusters IV and XIVa bacteria (e.g., non-Clostridia class bacteria), etc.

Some embodiments herein include testing the subject or patient for allergic reaction (e.g., allergen hypersensitivity) to one or more allergens (e.g., milk, milk proteins, eggs, fish, nuts from trees (e.g., hazelnuts, walnuts, almonds, Brazil nuts, etc.), peanuts (groundnuts), shellfish (e.g., shrimps, mussels, crab, etc.), soy, wheat, etc.). In some embodiments, a skin allergy test is performed to identify/confirm one or more allergen hypersensitivities in the subject. In some embodiments, a skin test is performed by a skin prick or scratch test, an intradermal skin test, and/or patch testing.

Some embodiments herein include testing the subject or patient for normal gut microbiota (e.g., microbiota that promotes allergen tolerance); abnormal gut microbiota (e.g., microbiota that promotes allergen hypersensitivity); or presence, absence, number, or relative abundance of specific taxa or strains of bacteria (e.g., non-Clostridia, Clostridia, etc.) in the gut microbiota. In some embodiments, such testing comprises: analysis of a biomarker such as a metabolite, nucleic acid, polypeptide, sugar, lipid, indication, symptom, etc. For example, in some embodiments the technology comprises testing using a labeled probe, a nucleic acid test (NAT), a nucleic acid amplification test (NAAT), a nucleic acid amplification technology (e.g., polymerase chain reaction (e.g., PCR, real-time PCR, probe hydrolysis PCR, reverse transcription PCR), isothermal amplification (e.g., nucleic acid sequence-based amplification (NASBA)), a ligase chain reaction, or a transcription mediated amplification, etc.), or nucleic acid sequencing (e.g., Sanger sequencing or next-gen (e.g., second generation, third generation, etc.) sequencing methods including, e.g., sequencing-by-synthesis, single molecule sequencing, nanopore, ion torrent, etc.).

Some embodiments comprise a second testing of the subject or patient (e.g., for microbiota composition, for allergen sensitivity, etc.), which may be the same or different from the first testing of the patient. In some embodiments, the second testing occurs after administration of a composition comprising non-Clostridium clusters IV and XIVa bacteria (e.g., non-Clostridia class bacteria (e.g., Acidaminococcus intestini, Alistipes putredinis, Bacteroides massiliensis, Bacteroides stercoris, Bifidobacterium adolescentis, Megamonas funiformis, Megamonas hypermegale, Megamonas rupellensis, and taxonomically-related bacteria that similarly modulate the immune response and/or support allergen tolerance)) to the subject. In some embodiments, the second testing indicates that the administration of the composition comprising n non-Clostridium clusters IV and XIVa bacteria (e.g., non-Clostridia class bacteria) to the subject is an effective treatment. In some embodiments, the second testing indicates that the administration of the composition comprising bacteria of the Clostridia class, including classes IV and/or XIVa, to the subject was an ineffective treatment. In some embodiments, the dose amount, dose schedule, and/or type or CFU of non-Clostridium clusters IV and XIVa bacteria (e.g., non-Clostridia class bacteria) in the composition is changed for subsequent administrations to the subject or patient based on the results of the test.

In some embodiments, methods comprise administering to a subject or patient a composition comprising non-Clostridium clusters IV and XIVa bacteria (e.g., non-Clostridia class bacteria) and a probiotic component or a prebiotic component.

The technology also comprises, in some embodiments, pharmaceutical compositions comprising non-Clostridium clusters IV and XIVa bacteria (e.g., non-Clostridia class bacteria) (e.g., spores, vegetative cells, etc.) and a pharmaceutically acceptable carrier. In some embodiments, the pharmaceutical composition comprises an effective amount of non-Clostridium clusters IV and XIVa bacteria (e.g., non-Clostridia class bacteria (e.g., Acidaminococcus intestini, Alistipes putredinis, Bacteroides massiliensis, Bacteroides stercoris, Bifidobacterium adolescentis, Megamonas funiformis, Megamonas hypermegale, Megamonas rupellensis, and taxonomically-related bacteria that similarly support immune modulation, resulting in reduced inflammatory conditions and/or allergen tolerance)). Also, in some embodiments, the pharmaceutical composition comprises additional components, e.g., in some embodiments the pharmaceutical composition comprises a probiotic or a prebiotic.

Non-limiting examples of prebiotics useful in the compositions and methods herein include xylose, arabinose, ribose, galactose, rhamnose, cellobiose, fructose, lactose, salicin, sucrose, glucose, esculin, tween 80, trehalose, maltose, mannose, mellibiose, raffinose, fructooligosaccharides (e.g., oligofructose, inulin, inulin-type fructans), galactooligosaccharides, amino acids, alcohols, water-soluble cellulose derivatives (most preferably, methylcellulose, methyl ethyl cellulose, hydroxyethyl cellulose, ethyl hydroxyethyl cellulose, cationic hydroxyethyl cellulose, hydroxypropyl cellulose, hydroxyethyl methylcellulose, hydroxypropyl methylcellulose, and carboxymethyl cellulose), water-insoluble cellulose derivatives (most preferably, ethyl cellulose), unprocessed oatmeal, metamucil, all-bran, and any combinations thereof.

Embodiments provide that pharmaceutical compositions are formulated for administration to a subject or a patient, e.g., some embodiments provide pharmaceutical compositions formulated for administration to a human. Some embodiments provide pharmaceutical compositions formulated for administration to a human newborn, neonate, infant, juvenile, teen, adult, or elderly patient. Related embodiments provide a pharmaceutical composition comprising live bacteria and/or bacterial spores from non-Clostridium clusters IV and XIVa bacteria (e.g., non-Clostridia class bacteria).

Embodiments provide pharmaceutical compositions formulated for various routes of administration, e.g., for providing the non-Clostridium clusters IV and XIVa bacteria (e.g., non-Clostridia class bacteria) to the gastrointestinal tract. For example, in some embodiments, the pharmaceutical composition is formulated for oral administration and in some embodiments the pharmaceutical composition is formulated for rectal administration. In some embodiments, the pharmaceutical composition is a nutraceutical or a food.

Related embodiments provide kits comprising a pharmaceutical composition comprising non-Clostridium clusters IV and XIVa bacteria (e.g., non-Clostridia class bacteria (e.g., Acidaminococcus intestini, Alistipes putredinis, Bacteroides massiliensis, Bacteroides stercoris, Bifidobacterium adolescentis, Megamonas funiformis, Megamonas hypermegale, Megamonas rupellensis, and taxonomically-related bacteria that similarly support immune modulation and/or allergen tolerance)) or as otherwise described herein. Some embodiments provide a kit for treating or preventing allergen hypersensitivity in a subject, the kit comprising a composition comprising non-Clostridium clusters IV and XIVa bacteria (e.g., non-Clostridia class bacteria) formulated for administration to the subject; and a reagent for testing the membership or relative abundance of one or more members of the gut microbiota of the subject. In some embodiments, the kit reagent comprises a labeled oligonucleotide probe. In some embodiments, the kit reagent comprises an amplification oligonucleotide. Embodiments of kits comprise a reagent that provides a test for the presence, absence, or level of non-Clostridium clusters IV and XIVa bacteria (e.g., non-Clostridia class bacteria) (e.g., specific strains or taxa described herein) and/or Clostridia class bacteria in the gut microbiota of the subject; a test for the membership or relative abundance of particular taxa or strains of bacterial in the gut microbiota of the subject; etc.

Some embodiments provide use of a composition comprising non-Clostridium clusters IV and XIVa bacteria (e.g., non-Clostridia class bacteria (e.g., Acidaminococcus intestini, Alistipes putredinis, Bacteroides massiliensis, Bacteroides stercoris, Bifidobacterium adolescentis, Megamonas funiformis, Megamonas hypermegale, Megamonas rupellensis, and taxonomically-related bacteria that similarly support allergen tolerance)) to treat a subject. Some embodiments provide use of a composition comprising bacteria of the non-Clostridium clusters IV and XIVa bacteria (e.g., non-Clostridia class bacteria) to manufacture a medicament for administration to a subject. Some embodiments provide use of a composition comprising bacteria of the non-Clostridium clusters IV and XIVa bacteria (e.g., non-Clostridia class bacteria) to treat or prevent inflammatory conditions and/or allergen hypersensitivity (e.g., to promote allergen tolerance, to promote allergen desensitization, etc.) in a subject.

Some embodiments provide a kit or system for treating or preventing allergen hypersensitivity in a subject, the system comprising a composition comprising non-Clostridium clusters IV and XIVa bacteria (e.g., non-Clostridia class bacteria) formulated for administration to the subject; and a reagent for testing the membership or relative abundance of one or more members of the gut microbiota of the subject.

In some embodiments, provided herein are pharmaceutical compositions comprising a bacteria and a pharmaceutically acceptable carrier, wherein the bacteria comprise a biologically pure culture of a strain from species: Megamonas funiformis, Megamonas hypermegale, Acidaminococcus intestine, Bacteroides massiliensis, Bacteroides stercoris, Alistipes putredinis, and/or Bifidobacterium adolescentis. In some embodiments, the bacteria comprise a biologically pure culture of: Megamonas funiformis DSM19343, Megamonas hypermegale DSM1672, Acidaminococcus intestine DSM21505, Bacteroides massiliensis DSM17679, Bacteroides stercoris ATCC43183/DSM19555, Alistipes putredinis DSM17216, and/or Bifidobacterium adolescentis ATCC15703. In some embodiments, the composition further comprises one or more (e.g., all) of: Faecalibacterium prausnitzii, Subdoligranulum variabile, Anaerostipes caccae, Marvinbryantia formatexigens, Clostridium scindens, and/or Ruminococcus bromii. In some embodiments, the composition comprises Faecalibacterium prausnitzii DSM17677, Subdoligranulum variabile DSM15176, Anaerostipes caccae DSM14662, Marvinbryantia formatexigens DSM14469, Clostridium scindens ATCC35704, and/or Ruminococcus bromii YE202.

In some embodiments, provided herein are pharmaceutical compositions comprising a bacteria and a pharmaceutically acceptable carrier, wherein the bacteria consists of a biologically pure culture of a strain from species: Megamonas funiformis, Megamonas hypermegale, Acidaminococcus intestine, Bacteroides massiliensis, Bacteroides stercoris, Alistipes putredinis, and Bifidobacterium adolescentis. In some embodiments, the bacteria comprise or consist of a biologically pure culture of: Megamonas funiformis DSM19343, Megamonas hypermegale DSM1672, Acidaminococcus intestine DSM21505, Bacteroides massiliensis DSM17679, Bacteroides stercoris ATCC43183/DSM19555, Alistipes putredinis DSM17216, and/or Bifidobacterium adolescentis ATCC15703. In some embodiments, compositions further comprise: Faecalibacterium prausnitzii, Subdoligranulum variabile, Anaerostipes caccae, Marvinbryantia formatexigens, Clostridium scindens, and/or Ruminococcus bromii. In some embodiments, compositions comprise: Faecalibacterium prausnitzii DSM17677, Subdoligranulum variabile DSM15176, Anaerostipes caccae DSM14662, Marvinbryantia formatexigens DSM14469, Clostridium scindens ATCC35704, and Ruminococcus bromii YE202.

In some embodiments, any of the aforementioned pharmaceutical compositions, further comprise a biologically pure culture of a strain of species Akkermansia muciniphila or Akkermansia muciniphila ATCC BAA-835.

### BRIEF DESCRIPTION OF THE DRAWINGS

FIG. 1. Principle Component Analysis based on weight unifrac distance metric for the taxonomic composition of genera assembled from the metagenomic data for all samples. The left circle represents infants that stayed sensitive to Cow's Milk following treatment (postEHCFLGG−), and the right circle identifies infants that gained tolerance to cow's milk (postEHCFLGG+).

FIGS. 2A and 2B. (A) Raw reads and contigs assigned to specific genus level taxa. (B) PCA analysis of only those samples covering infants that developed tolerance, but including both pre- and post-treatment with EHCFLGG.

FIGS. 3A and 3B. Analysis of genus level diversity for all sample types and identification of genus level taxa that are significantly more abundant in either the infants that became tolerant, or the infants that stayed sensitive to Cow's Milk protein. Butyrate producing strains are bolded.

FIGS. 4A and 4B. Increase in the relative proportion of Bifidobacterium and Clostridium in all treatment groups.

FIG. 5. Species phylogeny for taxa that were enriched in infants that became tolerant versus those that remained intolerant.

FIG. 6. Relative proportions of genes associated with Butyrate production across the four core treatment groups.

FIG. 7. Butyrate pathway genes that are significantly differentiated in abundance between children that developed tolerance versus those that remained sensitive.

FIG. 8. Butyrate pathways identified as being relevant in the predicted desensitization of infants. Schematic representation of pathways for carbohydrate fermentation in the large intestine. (1) Methanogenesis, (2) reductive acetogenesis, (3) butyryl CoA: acetate CoA transferase, (4) phosphotransbutyrylase/butyrate kinase, (5) phosphotransacetylase/acetate kinase, (6) lactate dehydrogenase, (7) acrylate pathway, and (8) succinate decarboxylation.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a non-Clostridia class bacteria strain” is a reference to one or more non-Clostridia class bacteria strains and equivalents thereof known to those skilled in the art, and so forth.

As used herein, the term “and/or” includes any and all combinations of listed items, including any of the listed items individually. For example, “A, B, and/or C” encompasses A, B, C, AB, AC, BC, and ABC, each of which is to be considered separately described by the statement “A, B, and/or C.” As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “subject” broadly refers to any animal, including but not limited to, human and non-human animals (e.g., dogs, cats, cows, horses, sheep, poultry (e.g., chickens), fish, crustaceans, etc.). As used herein, the term “patient” typically refers to a human subject that is being treated for a disease or condition.

As used herein, the term “infant”, when referring to a human, refers to a human between the ages of 1 month and 12 months.

As used herein, the term “newborn”, when referring to a human, is a human who is hours, days, or 1 to 3 weeks old.

As used herein, the term “neonate”, when referring to a human, refers to a newborn human and humans having an age up to and including 28 days after birth. The term “neonate” also refers to premature infants, postmature infants, and full term infants.

As used herein, the term “effective amount” refers to the amount of a composition sufficient to effect beneficial or desired results. An effective amount can be administered in one or more administrations, applications or dosages and is not intended to be limited to a particular formulation or administration route.

As used herein, the terms “administration” and “administering” refer to the act of giving a drug, prodrug, or other agent, or therapeutic treatment to a subject or in vivo, in vitro, or ex vivo cells, tissues, and organs. Exemplary routes of administration to the human body can be through space under the arachnoid membrane of the brain or spinal cord (intrathecal), the eyes (ophthalmic), mouth (oral), skin (topical or transdermal), nose (nasal), lungs (inhalant), oral mucosa (buccal), ear, rectal, vaginal, by injection (e.g., intravenously, subcutaneously, intratumorally, intraperitoneally, etc.) and the like.

As used herein, the terms “co-administration” and “co-administering” refer to the administration of at least two agent(s) (e.g., a pharmaceutical composition comprising non-Clostridium clusters IV and XIVa bacteria (e.g., non-Clostridia class bacteria), and/or additional therapeutics) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

As used herein, the term “pharmaceutical composition” refers to the combination of an active agent with a carrier, inert or active, making the composition especially suitable for diagnostic or therapeutic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintigrants (e.g., potato starch or sodium starch glycolate), and the like. The compositions also can include stabilizers and preservatives. For examples of carriers, stabilizers and adjuvants, see, e.g., Martin, Remington's Pharmaceutical Sciences, 15th Ed., Mack Publ. Co., Easton, Pa. (1975), incorporated herein by reference in its entirety.

As used herein, a “prebiotic” refers to an ingredient that allows specific changes, both in the composition and/or activity in the gastrointestinal microbiota that may (or may not) confer benefits upon the host. In some embodiments, a prebiotic is a comestible food or beverage or ingredient thereof. In some embodiments, a prebiotic is a selectively fermented ingredient. Prebiotics may include complex carbohydrates, amino acids, peptides, minerals, or other essential nutritional components for the survival of the bacterial composition. Prebiotics include, but are not limited to, amino acids, biotin, fructooligosaccharide, galactooligosaccharides, hemicelluloses (e.g., arabinoxylan, xylan, xyloglucan, and glucomannan), inulin, chitin, lactulose, mannan oligosaccharides, oligofructose-enriched inulin, gums (e.g., guar gum, gum arabic and carregenaan), oligofructose, oligodextrose, tagatose, resistant maltodextrins (e.g., resistant starch), trans-galactooligosaccharide, pectins (e.g., xylogalactouronan, citrus pectin, apple pectin, and rhamnogalacturonan-I), dietary fibers (e.g., soy fiber, sugarbeet fiber, pea fiber, corn bran, and oat fiber) and xylooligosaccharides.

As used herein, the term “microbe” refers to cellular prokaryotic and eukaryotic species from the domains Archaea, Bacteria, and Eukarya, the latter including yeast and filamentous fungi, protozoa, algae, or higher Protista, and encompasses both individual organisms and populations comprising any number of the organisms. The terms “microbial cells” and “microbes” are used interchangeably with the term “microorganism”.

The term “prokaryotes” refers to cells that contain no nucleus or other cell organelles. The prokaryotes are generally classified in one of two domains, the Bacteria and the Archaea. The definitive difference between organisms of the Archaea and Bacteria domains is based on fundamental differences in the nucleotide base sequence in the 16S ribosomal RNA. The terms “bacteria” and “bacterium” and “archaea” and “archaeon” refer to prokaryotic organisms of the domain Bacteria and Archaea in the three-domain system (see Woese C R, et al., Proc Natl Acad Sci USA 1990, 87: 4576-79).

As used herein, the term “phylogenetic tree” refers to a graphical or schematic representation of the evolutionary relationships of one genetic sequence to another that is generated, for example, using a defined set of phylogenetic reconstruction algorithms (e.g. parsimony, maximum likelihood, or Bayesian). Nodes in the tree represent distinct ancestral sequences and the confidence of any node is provided, for example, by a bootstrap or Bayesian posterior probability, which measures branch uncertainty.

“rDNA”, “rRNA”, “16S-rDNA”, “16S-rRNA”, “16S”, “16S sequencing”, “16S-NGS”, “18S”, “18S-rRNA”, “18S-rDNA”, “18S sequencing”, and “18S-NGS” refer to the nucleic acids that encode for the RNA subunits of the ribosome. rDNA refers to the gene that encodes the rRNA that comprises the RNA subunits. There are two RNA subunits in the ribosome termed the small subunit (SSU) and large subunit (LSU); the RNA genetic sequences (rRNA) of these subunits are related to the gene that encodes them (rDNA) by the genetic code. rDNA genes and their complementary RNA sequences are widely used for determination of the evolutionary relationships amount organisms as they are variable, yet sufficiently conserved to allow cross organism molecular comparisons. Typically 16S rDNA sequence (approximately 1542 nucleotides in length) of the 30S SSU is used for molecular-based taxonomic assignments of Prokaryotes and the 18S rDNA sequence (approximately 1869 nucleotides in length) of 40S SSU is used for Eukaryotes. 16S sequences are used for phylogenetic reconstruction as they are in general highly conserved, but contain specific hypervariable regions that harbor sufficient nucleotide diversity to differentiate genera and species of most bacteria. The “V1-V9 regions” of the 16S rRNA refers to the first through ninth hypervariable regions of the 16S rRNA gene that are used for genetic typing of bacterial samples. These regions in bacteria are defined by nucleotides 69-99, 137-242, 433-497, 576-682, 822-879, 986-1043, 1117-1173, 1243-1294 and 1435-1465 respectively using numbering based on the E. coli system of nomenclature (Brosius et al., PNAS 75(10):4801-4805 (1978); incorporated by reference in its entirety). In some embodiments, at least one of the V1, V2, V3, V4, V5, V6, V7, V8, and V9 regions are used to characterize an OTU. In one embodiment, the V1, V2, and V3 regions are used to characterize an OTU. In another embodiment, the V3, V4, and V5 regions are used to characterize an OTU. In another embodiment, the V4 region is used to characterize an OTU. A person of ordinary skill in the art can identify the specific hypervariable regions of a candidate 16S rRNA by comparing the candidate sequence in question to a reference sequence and identifying the hypervariable regions based on similarity to the reference hypervariable regions, or alternatively, one can employ Whole Genome Shotgun (WGS) sequence characterization of microbes or a microbial community.

As used herein, the term “operational taxonomic units” (“OTU”) refers to a terminal leaf in a phylogenetic tree and is defined by a nucleic acid sequence, e.g., the entire genome, or a specific genetic sequence, and all sequences that share sequence identity to this nucleic acid sequence at the level of species. In some embodiments the specific genetic sequence may be the 16S sequence or a portion of the 16S sequence. In other embodiments, the entire genomes of two entities are sequenced and compared. In another embodiment, select regions such as multilocus sequence tags (MLST), specific genes, or sets of genes may be genetically compared. In some embodiments, OTUs that share ≥97% average nucleotide identity across the entire 16S or some variable region of the 16S are considered the same OTU (see e.g. Claesson et al. 2010. Nucleic Acids Res 38: e200.; Konstantinidis et al. 2006. Philos Trans R Soc Lond B Biol Sci 361: 1929-1940.; incorporated by reference in their entireties). In embodiments involving the complete genome, MLSTs, specific genes, or sets of genes OTUs that share ≥95% average nucleotide identity are considered the same OTU (see e.g. Achtman M, and Wagner M. 2008. Nat. Rev. Microbiol. 6: 431-440.; Konstantinidis et al. 2006. Philos Trans R Soc Lond B Biol Sci 361: 1929-1940.; incorporated by reference in their entireties). OTUs are frequently defined by comparing sequences between organisms. Generally, sequences with less than 95% sequence identity are not considered to form part of the same OTU. OTUs may also be characterized by any combination of nucleotide markers or genes, in particular highly conserved genes (e.g., “house-keeping” genes), or a combination thereof. Such characterization employs, e.g., WGS data or a whole genome sequence.

The term “genus” is defined as a taxonomic group of related species according to the Taxonomic Outline of Bacteria and Archaea (Garrity et al. (2007) The Taxonomic Outline of Bacteria and Archaea. TOBA Release 7.7, March 2007. Michigan State University Board of Trustees).

The term “species” is defined as collection of closely related organisms with greater than 97% 16S ribosomal RNA sequence homology and greater than 70% genomic hybridization and sufficiently different from all other organisms so as to be recognized as a distinct unit (e.g., an operational taxonomic unit).

The term “strain” as used herein in reference to a microorganism describes an isolate of a microorganism considered to be of the same species but with a unique genome and, if nucleotide changes are non-synonymous, a unique proteome differing from other strains of the same organism. Strains may differ in their non-chromosomal genetic complement. Typically, strains are the result of isolation from a different host or at a different location and time, but multiple strains of the same organism may be isolated from the same host.

As used herein, the term “microbiota” refers to an assemblage of microorganisms localized to a distinct environment. Microbiota may include, for example, populations of various bacteria, eukaryotes (e.g., fungi), and/or archaea that inhabit a particular environment. For example, “gut microbiota,” “vaginal microbiota,” and “oral microbiota” refer to an assemblage of one or more species of microorganisms that are localized to, or found in, the gut, vagina, or mouth, respectively.

“Normal microbiota” refers to a population of microorganisms that localize in a particular environment in a normal, non-pathological state (e.g., a sample of gut microbiota from a subject without an allergen hypersensitivity). A “normal microbiota” has normal membership and normal relative abundance.

“Abnormal microbiota” refers to a population of various microorganisms that localize in a particular environment in a subject suffering from or at risk of a pathological condition (e.g., a sample of gut microbiota from a subject with an allergen hypersensitivity). Abnormal microbiota differs from normal microbiota in terms of identity (e.g., membership), absolute amount, or relative amount (e.g., relative abundance) of the various microbes.

As used herein, the term “commensal microbe” refers to a microorganism that is non-pathogenic to a host and is part of the normal microbiota of the host.

As used herein, the terms “microbial agent,” “commensal microbial agent,” and “probiotic” refer to compositions comprising a microbe or population of multiple different microbes for administration to a subject.

The term “biosynthetic pathway”, also referred to as “metabolic pathway”, refers to a set of anabolic or catabolic biochemical reactions for converting one chemical species into another. Gene products belong to the same “metabolic pathway” if they, in parallel or in series, act on the same substrate, produce the same product, or act on or produce a metabolic intermediate (e.g., a metabolite) between the same substrate and metabolite end product.

As used herein, the term “taxonomic unit” is a group of organisms that are considered similar enough to be treated as a separate unit. A taxonomic unit may comprise, e.g., a class, family, genus, species, or population within a species (e.g., strain), but is not limited as such.

As used herein, the terms “operation taxonomic unit,” “OTU,” and “taxon” are used interchangeably to refer to a group of microorganisms considered similar enough to be treated as a separate unit. In one embodiment, an OTU is a group tentatively assumed to be a valid taxon for purposes of phylogenetic analysis. In another embodiment, an OTU is any of the extant taxonomic units under study. In yet another embodiment, an OTU is given a name and a rank. For example, an OTU can represent a domain, a sub-domain, a kingdom, a sub-kingdom, a phylum, a sub-phylum, a class, a sub-class, an order, a sub-order, a family, a subfamily, a genus, a subgenus, a species, a subspecies, a strain, etc. In some embodiments, OTUs can represent one or more organisms from the domains Bacteria, Archaea, or Eukarya at any level of a hierarchal order. In some embodiments, an OTU represents a prokaryotic or fungal order. In some embodiments, an OTU is defined based on extent of homology between biomolecular (e.g., nucleic acid, polypeptide) sequences (e.g., percent identity). For example, in certain cases, the OTU may include a group of microorganisms treated as a unit based on, e.g., a sequence identity of ≥95%, ≥90%, ≥80%, or ≥70% among at least a portion of a differentiating biomarker, e.g., a biomolecule such as the 16S rRNA gene.

As used herein, a “colony-forming unit” (“CFU”) is used as a measure of viable microorganisms in a sample. A CFU is an individual viable cell capable of forming on a solid medium a visible colony whose individual cells are derived by cell division from one parental cell.

As used herein, the term “relative abundance” relates to the abundance of microorganisms of a particular taxonomic unit or OTU in a test biological sample compared to the abundance of microorganisms of the corresponding taxonomic unit or OTU in one or more non-diseased control samples. The “relative abundance” may be reflected in e.g., the number of isolated species corresponding to a taxonomic unit or OTU or the degree to which a biomarker specific for the taxonomic unit or OTU is present or expressed in a given sample. The relative abundance of a particular taxonomic unit or OTU in a sample can be determined using culture-based methods or non-culture-based methods well known in the art. Non-culture based methods include sequence analysis of amplified polynucleotides specific for a taxonomic unit or OTU or a comparison of proteomics-based profiles in a sample reflecting the number and degree of polypeptide-based, lipid-based, polysaccharide-based or carbohydrate-based biomarkers characteristic of one or more taxonomic units or OTUs present in the samples. Relative abundance or abundance of a taxon or OTU can be calculated with reference to all taxa/OTUs detected, or with reference to some set of invariant taxa/OTUs.

Methods for profiling the relative abundances of microbial taxa in biological samples, including biological samples of gut microbiota, are well known in the art. Suitable methods may be sequencing-based or array-based. For example, the microbial component of a gut microbiota sample is characterized by sequencing a nucleic acid suitable for taxonomic classification and assigning the sequencing reads to operational taxonomic units (OTUs) with a defined (e.g., >97%) nucleotide sequence identity to a database of annotated and representative sequences. An example of such a database is Greengenes version of May 2013; however any suitable database may be used. After OTUs are defined, a representative sequence from each OTU can be selected and compared to a reference set. If a match is identified in the reference set, that OTU can be given an identity. Relative abundance of a bacterial taxon may be defined by the number of sequencing reads that can be unambiguously assigned to each taxon after adjusting for genome uniqueness. Other methods of profiling the relative abundances of microbial taxa in biological samples are known within the field and within the scope herein.

In some embodiments, a suitable nucleic acid for taxonomic classification is universally distributed among the gut microbial population being queried allowing for the analysis of phylogenetic relationships among distant taxa, and has both a conserved region and at least one region subject to variation. The presence of at least one variable region allows sufficient diversification to provide a tool for classification, while the presence of conserved regions enables the design of suitable primers for amplification (if needed) and/or probes for hybridization for various taxa at different taxonomic levels ranging from individual strains to whole phyla. While any suitable nucleic acid known in the art may be used, one skilled in the art will appreciate that selection of a nucleic acid or region of a nucleic acid to amplify may differ by environment. In some embodiments, a nucleic acid queried is a small subunit ribosomal RNA gene. For bacterial and archaeal populations, at least the V1, V2, V3, V4, V5, V6, V7, V8, and/or V9 regions of the 16S rRNA gene are suitable, though other suitable regions are known in the art. Guidance for selecting a suitable 16S rRNA region to amplify can be found throughout the art, including Guo et al. PLOS One 8(10) e76185, 2013; Soergel D A W et al. ISME Journal 6: 1440, 2012; and Hamady M et al. Genome Res. 19:1 141, 2009, each hereby incorporated by reference in its entirety.

As used herein, the term “Clostridia” refers to a polyphyletic class of Firmicutes, including Clostridium and other similar genera. Clostridia are obligate anaerobes and are often but not always Gram-positive; some Clostridia form spores. In some embodiments, the term “Clostridia” refers to organisms in the taxonomic order Clostridiales. Clostridia are classified according cluster numbers (e.g., I through XIX).

As used herein, the term “non-Clostridia class bacteria” refers to bacteria that are not members of the class Clostridia, described above. Non-Clostridia class bacteria may be of other classes of Firmicutes, such as bacteria of the class Negativicutes (e.g. Megamonas, Acidaminococcus, Succinispira, Megasphaera, Dialister, Pelosiunus, Veillonella, etc.); or may be of other phyla, such as the plylum Bacteroidetes, including bacteria of the class Bacteroidia (e.g. Rikenella, Alistipes, Anaerocella, Porphyromonas, Prevotella, Hallella, Alloprevotella, etc.).

As used herein, the term “non-Clostridium clusters IV and XIVa” refers to bacteria (e.g., Clostridia or non-Clostridia) that are not part of either Clostridium cluster IV or XIVa. All non-Clostridia bacteria are non-Clostridium clusters IV and XIVa, and any Clostridia bacteria that are part of other clusters are non-Clostridium clusters IV and XIVa bacteria.

As used herein, the term “extensively hydrolyzed casein formula” (“EHCF”) refers to infant formula in which the protein components have been hydrolyzed to sufficient degree such that most of the nitrogen is in the form of free amino acids and peptides <1500 kDa. EHCFs have been used for >50 years for feeding infants with severe inflammatory bowel diseases or cow's milk allergies, and more recently to prevent the development of allergies in infants at high risk for developing allergic symptoms.

### DETAILED DESCRIPTION

Provided herein are compositions (e.g., probiotic, pharmaceutical, etc.) comprising one or more strains of non-Clostridium clusters IV and XIVa bacteria (e.g., non-Clostridia class bacteria) and methods of use thereof for allergen desensitization. In particular, bacteria of bacterial classes such as Negativicutes, Actinobacteria and Bacteroidia support allergen desensitization, for example, by promoting production of metabolites that aid in desensitization, performing catabolism of allergens (e.g., food allergens), or by providing essential nutrients to Clostridium clusters IV and XIVa bacteria, thereby promoting the activity of these bacteria that are also underrepresented in subjects suffering from inflammation, including food allergies.

Experiments conducted during development of embodiments herein indicated that other helper strains of bacteria (e.g., Acidaminococcus intestini, Alistipes putredinis, Bacteroides massiliensis, Bacteroides stercoris, Bifidobacterium adolescentis, Megamonas funiformis, Megamonas hypermegale, Megamonas rupellensis, and taxonomically-related bacteria that similarly support allergen tolerance) are important for enabling allergen desensitization. In some embodiments, organisms belonging to the classes Negativicutes (e.g. Megamonas, Acidaminococcus, Succinispira, Megasphaera, Dialister, Pelosiunus, Veillonella, etc.) and Bacteroidia (e.g. Rikenella, Alistipes, Anaerocella, Porphyromonas, Prevotella, Hallella, Alloprevotella, etc.) support desensitization by (1) supporting butyrate production by organisms belonging to the Clostridia classes IV and XIVa (and therefore activate regulatory T cell (Treg) accumulation); (2) produce other metabolites (e.g. Propionate) that aid in desensitization and immune modulation; (3) cause a decrease in the secretion of a pro-inflammatory cytokine or an enhanced secretion of an anti-inflammatory cytokine by a population of human peripheral blood mononuclear cells at levels sufficient to allow for immune response modulation (4) perform catabolism of allergens (e.g., food allergens), thereby reducing the impact of allergens on sensitization; and/or (5) support or play a role in other useful metabolic pathways, e.g. indole that plays a key role in tightening the junctions between the epithelial cells that line the gut, thereby reducing leakage of inflammatory antigens into the blood stream; however, embodiments herein is not limited to any particular mechanism of action and an understanding of the mechanism of action is not necessary- to practice such embodiments.

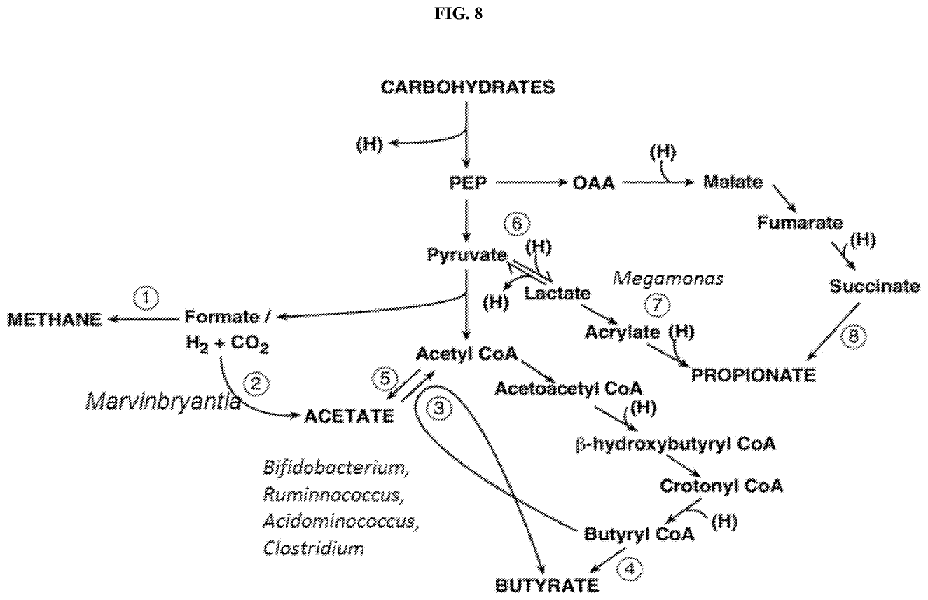

Supplementing the diet of cow's-milk allergic infants with extensive hydrolyzed casein formula and Lactobacillus rhamnosus GG substantial improves desensitization outcomes. This treatment led to substantially different microbial community composition, and that microbiome composition differs significantly between infants that develop tolerance and those that don't. An enrichment of Blautia, Roseburia and Coprococcus (all Clostridia Cluster XIVa) is observed in treated infants, but only Oscillospira (Clostridia Cluster XIVa) is enriched in infants that developed tolerance. Experiments were conducted during development of embodiments herein to sequenced a relevant subset of samples using shotgun metagenomics techniques, and the genomes of all organisms, as well as the metabolic pathways, were reconstructed. Based on a comparative microbiome analysis, a defined consortium of human-derived keystone species was identified in which the experiments indicate that the consortium restores gut health and modulates the immune response. In some embodiments, this consortium of bacteria, or subcombinations thereof, finds use in the treatment of allergies (e.g., food allergies (e.g., milk allergies, etc.), etc.) or desensitization of subjects to allergens, either alone or with other treatments. In some embodiments, this consortium comprises species of Bifidobacterium, Ruminnococcus, Acidominococcus, Alistipes and Megamonas. In some embodiments, human-isolated taxa including Acidaminococcus intestini, Alistipes putredinis, Bacteroides massiliensis, Bacteroides stercoris, Bifidobacterium adolescentis, Megamonas funiformis, Megamonas hypermegale, Megamonas rupellensis, Megamonas unclassified (new species), and Ruminococcus gnavis support immune activation, Treg recruitment, and reduce inflammation. In some embodiments, the inclusion of Blautia hydrogenotrophica and Marvinbryantia formatexigens, which are acetogenic, supplies the butyrate production pathways with acetate, that support increased butyrate activity, while reducing the concentrations of formate, which has an inflammatory effect. Butyryl CoA-Acetyl CoA transferase drives the production of Acetyl CoA from acetate and is mediated by the Type XIVa Clostridia (FIG. 8). In some embodiments, Megamonas species mediate the production of propionate from pyruvate. In some embodiments, this pathway is upregulated in tolerant subjects, but not in allergen-sensitive subjects. In some embodiments, propionate also stimulates Treg accumulation. In some embodiments, reductive acetogenesis is mediated by Marvinbryantia formatexigens, which breaks down formate to produce acetate (e.g., as a byproduct of removing hydrogen), which feeds the acetate cycle to produce butyrate.

In some embodiments, administration of certain non-Clostridium clusters IV and XIVa bacteria (e.g., non-Clostridia class bacteria) facilitates establishment of gut microbiota that is beneficial to (1) the desensitization of a subject to allergens (e.g., milk), or (2) the prevention of the development of sensitivity (e.g., hypersensitivity) to allergens (e.g., milk). In some embodiments, establishing beneficial microbiota creates an environment (e.g., through the production of gene products and metabolites) to decrease or prevent (chronic) inflammatory conditions, and/or desensitizes a subject to allergens (e.g., milk) or prevent sensitization (e.g., hypersensitization) of the subject to allergens (e.g., milk). Accordingly, compositions, kits, systems are provided comprising non-Clostridium clusters IV and XIVa bacteria (e.g., non-Clostridia class bacteria), and methods of use thereof for the treatment and/or prevention of inflammatory conditions and/or allergen hypersensitivity.

Accordingly, in some embodiments, the present technology provides compositions and kits, e.g., for administration to a subject. In some embodiments, compositions comprise one or more non-Clostridium clusters IV and XIVa (e.g., non-Clostridia class) bacterial species. Embodiments are not limited to a particular one or more bacterial species. Examples include, but are not limited to, those described herein (e.g., from the classes Negativicutes, Actinobacteria, Bacteroidia, etc.).

In some embodiments, compositions and kits comprise bacteria that support butyrate production, activate Treg accumulation, produce or support the production of propionate, catabolize allergens, support enrichment of Oscillospira (Clostridia Cluster XIVa), cause a decrease in the secretion of a pro-inflammatory cytokine or an enhanced secretion of an anti-inflammatory cytokine by a population of human peripheral blood mononuclear cells at levels sufficient to allow for immune response modulation, etc. Composition may comprise a single classification (e.g., strain, species, genus, etc.) of bacteria, or multiple classifications of bacteria.

In some embodiments, bacteria are selected from the phyla Actinobacteria (e.g., genus Bifidobacterium), Bacteroidetes (e.g., Rikenella, Alistipes, Anaerocella, Porphyromonas, Prevotella, Hallella, Alloprevotella genus, etc.), and Firmicutes (e.g., non-Clostridia class, and Negativicutes (e.g. Megamonas, Acidaminococcus, Succinispira, Megasphaera, Dialister, Pelosiunus, Veillonella, etc.), etc.).

In some embodiments, compositions/kits comprise a single species of bacteria. In other embodiments, the compositions/kits comprise two or more species of bacteria, e.g., 2, 3, 4, 5, 6, 7, 8, 9, 10, 15, 20, 30, 40, 50, 60, 70, 80, 90, 100, 500, 1000 or more, or ranges therebetween species of bacteria. In one embodiment, compositions/kits comprise no more than 20 species of bacteria, e.g., 20, 19, 18, 17, 16, 15, 14, 13, 12, 11, 10, 9, 8, 7, 6, 5, 4, 3, 2, or 1 species of bacteria. In some embodiments, methods of administering such compositions/kits are provided.

In some embodiments, compositions/kits comprise a single OTU. In some embodiments, compositions/kits comprise two or more OTUs (e.g., 2, 3, 4, 5, 6, 7, 8, 9, 10, 15, 20, 30, 40, 50, 60, 70, 80, 90, 100, 500, 1000 or more, or ranges therebetween). In some embodiments, each OTU independently characterized by, for example, at least 95%, 96%, 97%, 98%, 99% 100% sequence identity a reference sequence (e.g., a segment of 16S RNA) for the OTU (e.g., species, genus, etc.).

In some embodiments, compositions comprise one or more bacteria species/strains from the Actinobacteria phylum. In some embodiments, Actinobacteria are of the genus Bifidobacterium. In some embodiments, Bifidobacteria are Bifidobacterium adolescentis.

In some embodiments, compositions comprise one or more bacteria species/strains from the Bacteroidetes phylum. In some embodiments, Bacteroidetes are of the class Bacteroidia. In some embodiments, Bacteroidetes are of the genus Rikenella, Alistipes, Anaerocella, Porphyromonas, Prevotella, Hallella, and/or Alloprevotella. In some embodiments, compositions comprise one or more bacteria species/strains from the Firmicutes phylum. In some embodiments, Firmicutes are of the class Negativicutes.

In some embodiments, a composition or kit comprises microbes from one or more bacterial families within the class Negativicutes, such as: Selenomonadaceae (e.g., genus: Anaerovibrio, Centipeda, Megamonas, Mitsuokella, Pectinatus, Propionispira, Schwartzia, Selenomonas, Zymophilus), Acidaminococcaceae (e.g., genus: Acidaminococcus, Phascolarctobacterium, Succiniclasticum, Succinispira, etc.), Sporomusaceae (e.g., genus: Acetonema, Anaeroarcus, Anaeromusa, Anaerosinus, Anaerospora, Dendrosporobacter, Desulfosporomusa, Pelosinus, Propionispora, Psychrosinus, Sporolituus, Sporomusa, Thermosinus), Veillonellaceae (e.g., genus: Allisonella, Anaeroglobus, Dialister, Megasphaera, Negativicoccus, Veillonella), etc.

In some embodiments, a composition or kit comprises microbes from one or more bacterial families within the class Bacteroidia (and/or order Bacteroidales), such as: Bacteroidaceae (e.g., genus: Bacteroides, Acetofilamentum, Acetomicrobium, Acetothermus, Anaerorhabdus, etc.), Marinilabiliaceae (e.g., genus: Alkaliflexus, Alkalitalea, Anaerophaga, Geofilum, Mangroviflexus, Marinilabilia, Natronoflexus, Thermophagus, etc.), Porphyromonadaceae (e.g., genus: Porphyromonas, Dysgonomonas, etc.), Prolixibacteraceae, Prevotellaceae (e.g., genus: Prevotella, Alloprevotella, Hallella, Paraprevotella, etc.), Rikenellaceae (e.g., Rikenella, Alistipes, Anaerocella, etc.), etc.

In some embodiments, a composition or kit comprises one or more bacterial species selected from Acidaminococcus intestini, Alistipes putredinis, Bacteroides massiliensis, Bacteroides stercoris, Bifidobacterium adolescentis, Megamonas funiformis, Megamonas hypermegale, Megamonas rupellensis, and/or Ruminococcus gnavis.

In some embodiments, a composition or kit comprises one or more bacterial species from the family Bacteroidaceae and genus Bacteroides, such as, B. acidifaciens, B. distasonis (reclassified as Parabacteroides distasonis), B. gracilis, B. fragilis, B. oris, B. ovatus, B. putredinis, B. pyogenes, B. stercoris, B. suis, B. tectus, B. thetaiotaomicron, B. vulgatus, etc. In some embodiments, a composition or kit comprises one or more bacterial species from the family Bacteroidaceae and genus Acetofilamentum, such as, A. rigidum. In some embodiments, a composition or kit comprises one or more bacterial species from the family Bacteroidaceae and genus Acetomicrobium, such as, A. flavidum. In some embodiments, a composition or kit comprises one or more bacterial species from the family Bacteroidaceae and genus Acetothermus, such as, A. paucivorans. In some embodiments, a composition or kit comprises one or more bacterial species from the family Bacteroidaceae and genus Anaerorhabdus, such as, Anaerorhabdus furcosa.

In some embodiments, compositions comprise one or more additional components (e.g., including but not limited to, one or more additional additive(s) selected from the group consisting of an energy substrate, a mineral, a vitamin, or combinations thereof).

In some embodiments, in addition to non-Clostridia class bacteria, a composition or kit comprises one or more bacteria selected from the class Clostridia. For example, in some embodiments, in addition to non-Clostridia class bacteria, one or more bacteria species of the taxonomic order Clostridiales are administered, such as those from the taxonomic families: Caldicoprobacteraceae, Christensenellaceae, Clostridiaceae, Defluviitaleaceae, Eubacteriaceae, Graciibacteraceae, Heliobacteriaceae, Lachnospiraceae, Oscillospiraceae, Peptococcaceae, Peptostreptococcaceae, Ruminococcaceae, Syntrophomonadaceae, and Veillonellaceae. In some embodiments, Clostridia class bacteria are of the genus Ruminococcus. In some embodiments, a Ruminococcus is Ruminococcus gnavus. In some embodiments, Clostridia class bacteria are selected from Blautia hydrogenotrophica and/or Marvinbryantia formatexigens.

In some embodiments, bacteria are vegetative cells, freeze-dried cells, where possible spores, etc. Freeze-dried bacteria can be stored for several years with maintained viability. In certain applications, freeze-dried bacteria are sensitive to humidity. One way of protecting the bacterial cells is to store them in oil. The freeze dried bacterial cells can be mixed directly with a suitable oil, or alternately the bacterial cell solution can be mixed with an oil and freeze dried together, leaving the bacterial cells completely immersed in oil. Suitable oils may be edible oils such as olive oil, rapeseed oil which is prepared conventionally or cold-pressed, sunflower oil, soy oil, maize oil, cotton-seed oil, peanut oil, sesame oil, cereal germ oil such as wheat germ oil, grape kernel oil, palm oil and palm kernel oil, linseed oil. The viability of freeze-dried bacteria in oil is maintained for at least nine months. Optionally live cells can be added to one of the above oils and stored.

In some embodiments, the compositions are part of a milk replacer (e.g., for administration to a neonatal or young animal). In some embodiments, compositions comprise one or more bacteria as described herein in combination with a EHCF or a formula not derived from milk.

In some embodiments, compositions are added to nutraceuticals, food products, or foods. In some embodiments, to give the composition or nutraceutical a pleasant taste, flavoring substances such as for example mints, fruit juices, licorice, Stevia rebaudiana, steviosides or other calorie free sweeteners, rebaudioside A, essential oils like eucalyptus oil, or menthol can optionally be included in compositions of embodiments of the present invention.

In some composition embodiments, compositions are formulated in pharmaceutical compositions. The bacteria of embodiments herein may be administered alone or in combination with pharmaceutically acceptable carriers or diluents, and such administration may be carried out in single or multiple doses as described herein.

Compositions may, for example, be in the form of tablets, resolvable tablets, capsules, bolus, drench, pills sachets, vials, hard or soft capsules, aqueous or oily suspensions, aqueous or oily solutions, emulsions, powders, granules, syrups, elixirs, lozenges, reconstitutable powders, liquid preparations, creams, troches, hard candies, sprays, chewing-gums, creams, salves, jellies, gels, pastes, toothpastes, rinses, dental floss and tooth-picks, liquid aerosols, dry powder formulations, HFA aerosols or organic or inorganic acid addition salts.

The pharmaceutical compositions of embodiments of the invention may be in a form suitable for, e.g., rectal, oral, topical, buccal administration. Depending upon the disorder and patient to be treated and the route of administration, the compositions may be administered at varying doses.

In some embodiments, one or more non-Clostridium clusters IV and XIVa bacteria (e.g., non-Clostridia class bacteria) (e.g., alone or with other active components) are formulated in pharmaceutical compositions for rectal administration. Such formulations include enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

In some embodiments, one or more non-Clostridium clusters IV and XIVa bacteria (e.g., non-Clostridia class bacteria) (e.g., alone or with other active components) are formulated in pharmaceutical compositions for oral administration. Oral dosage forms include push fit capsules made of gelatin, as well as soft, sealed capsules made of gelatin and a plasticizer, such as glycerol or sorbitol. In specific embodiments, push fit capsules contain the active ingredients in admixture with one or more filler. Fillers include, by way of example only, lactose, binders such as starches, and/or lubricants such as talc or magnesium stearate and, optionally, stabilizers. In other embodiments, soft capsules, contain one or more active compound that is dissolved or suspended in a suitable liquid. Suitable liquids include, by way of example only, one or more fatty oil, liquid paraffin, or liquid polyethylene glycol. In addition, stabilizers are optionally added.

In some embodiments, the bacterial formulation comprises at least 1×10⁴ CFU (e.g., 1×10⁴ CFU, 2×10⁴ CFU, 5×10⁴ CFU, 1×10⁵ CFU, 2×10⁵ CFU, 5×10⁵ CFU, 1×10⁶ CFU, 2×10⁶ CFU, 5×10⁶ CFU, 1×10⁷ CFU, 2×10⁷ CFU, 5×10⁷ CFU, 1×10⁸ CFU, 2×10⁸ CFU, 5×10⁸ CFU, 1×10⁹ CFU, 2×10⁹ CFU, 5×10⁹ CFU, 1×10¹⁹ CFU, 2×10¹⁹ CFU, 5×10¹⁰ CFU, 1×10¹¹ CFU, 2×10¹¹ CFU, 5×10¹¹ CFU, 1×10¹² CFU, 2×10¹² CFU, 5×10¹² CFU, or more or ranges there between) of non-Clostridium clusters IV and XIVa bacteria (e.g., non-Clostridia class bacteria) (e.g., from a single classification (e.g., strain, species, genus, family, class, etc.), or from multiple non-Clostridium clusters IV and XIVa (e.g., non-Clostridia class) classifications). In some embodiments, the bacterial formulation is administered to the subject in two or more doses (e.g., 2, 3, 4, 5, 6, 7, 8, 9, 10, or more, or ranges there between). In some embodiments, the administration of doses are separated by at least 1 day (e.g., 2 days, 3 days, 4 days, 5 days, 6 days, 1 week, 2 weeks, 3 weeks, 4 weeks, or ranges there between).

For oral or buccal administration, bacteria of embodiments of the present invention may be combined with various excipients. Solid pharmaceutical preparations for oral administration often include binding agents (for example syrups, acacia, gelatin, tragacanth, polyvinylpyrrolidone, sodium lauryl sulphate, pregelatinized maize starch, hydroxypropyl methylcellulose, starches, modified starches, gum acacia, gum tragacanth, guar gum, pectin, wax binders, microcrystalline cellulose, methylcellulose, carboxymethylcellulose, hydroxypropyl methylcellulose, hydroxyethyl cellulose, hydroxypropyl cellulose, copolyvidone and sodium alginate), disintegrants (such as starch and preferably corn, potato or tapioca starch, alginic acid and certain complex silicates, polyvinylpyrrolidone, gelatin, acacia, sodium starch glycollate, microcrystalline cellulose, crosscarmellose sodium, crospovidone, hydroxypropyl methylcellulose and hydroxypropyl cellulose), lubricating agents (such as magnesium stearate, sodium lauryl sulfate, talc, silica polyethylene glycol waxes, stearic acid, palmitic acid, calcium stearate, carnuba wax, hydrogenated vegetable oils, mineral oils, polyethylene glycols and sodium stearyl fumarate) and fillers (including high molecular weight polyethylene glycols, lactose, calcium phosphate, glycine magnesium stearate, starch, rice flour, chalk, gelatin, microcrystalline cellulose, calcium sulphate, and lactitol). Such preparations may also include preservative agents and anti-oxidants.

Liquid compositions for oral administration may be in the form of, for example, emulsions, syrups, or elixirs, or may be presented as a dry product for reconstitution with water or other suitable vehicle before use. Such liquid compositions may contain conventional additives such as suspending agents (e.g. syrup, methyl cellulose, hydrogenated edible fats, gelatin, hydroxyalkylcelluloses, carboxymethylcellulose, aluminium stearate gel, hydrogenated edible fats) emulsifying agents (e.g. lecithin, sorbitan monooleate, or acacia), aqueous or non-aqueous vehicles (including edible oils, e.g. almond oil, fractionated coconut oil) oily esters (for example esters of glycerine, propylene glycol, polyethylene glycol or ethyl alcohol), glycerine, water or normal saline; preservatives (e.g. methyl or propyl p-hydroxybenzoate or sorbic acid) and conventional flavoring, preservative, sweetening or coloring agents. Diluents such as water, ethanol, propylene glycol, glycerin and combinations thereof may also be included.

Other suitable fillers, binders, disintegrants, lubricants and additional excipients are well known to a person skilled in the art.

In some embodiments, microbes are spray-dried. In other embodiments, microbes are suspended in an oil phase and are encased by at least one protective layer, which is water-soluble (water-soluble derivatives of cellulose or starch, gums or pectins; See e.g., EP 0 180 743, herein incorporated by reference in its entirety). In some embodiments, the present technology provides kits, pharmaceutical compositions, or other delivery systems for use in treatment or prevention of allergen hypersensitivity in a subject. The kit may include any and all components necessary, useful or sufficient for research or therapeutic uses including, but not limited to, one or more non-Clostridium clusters IV and XIVa (e.g., non-Clostridia class) microbes, pharmaceutical carriers, and additional components useful, necessary or sufficient for use in treatment (desensitization) or prevention (preventing development of allergen hypersensitivity) of allergen hypersensitivity. In some embodiments, the kits provide a sub-set of the required components, wherein it is expected that the user will supply the remaining components. In some embodiments, the kits comprise two or more separate containers wherein each container houses a subset of the components to be delivered.

Optionally, compositions and kits comprise other active components in order to achieve desired therapeutic effects.

In some embodiments, compositions and kits provided herein (e.g., comprising non-Clostridium clusters IV and XIVa bacteria (e.g., non-Clostridia class bacteria)) are administered once to a subject in need thereof.

In some embodiments, compositions comprise prebiotic compounds such as carbohydrate compounds selected from the group consisting of inulin, fructooligosaccharide (FOS), short-chain fructooligosaccharide (short chain FOS), galacto-oligosaccharide (GOS), xylooligosaccharide (XOS), glangliosides, partially hydrolysed guar gum (PHGG) acacia gum, soybean-gum, apple extract, lactowolfberry, wolfberry extracts or mixture thereof. Other carbohydrates may be present such as a second carbohydrate acting in synergy with the first carbohydrate and that is selected from the group consisting of xylooligosaccharide (XOS), gum, acacia gum, starch, partially hydrolysed guar gum or mixture thereof. The carbohydrate or carbohydrates may be present at about 1 g to 20 g or 1% to 80% or 20% to 60% in the daily doses of the composition. Alternatively, the carbohydrates are present at 10% to 80% of the dry composition.

The daily doses of carbohydrates, and all other compounds administered with the probiotics comply with published safety guidelines and regulatory requirements. This is particularly important with respect to the administration to newborn babies.

In some embodiments, a nutritional composition preferably comprises a source of protein. Dietary protein is preferred as a source of protein. The dietary protein may be any suitable dietary protein, for example animal proteins, vegetable proteins (such as soy proteins, wheat proteins, rice proteins or pea proteins), a mixture of free amino acids, or a combination thereof. In some embodiments, milk proteins such as casein and whey proteins are avoided. The composition may also comprise a source of carbohydrates and/or a source of fat.

In some embodiments, compositions are administered on an ongoing, recurrent, or repeat basis (e.g., multiple times a day, once a day, once every 2, 3, 4, 5, or 6 days, once a week, etc.) for a period of time (e.g., multiple days, months, or weeks). Suitable dosages and dosing schedules are determined by one of skill in the art using suitable methods.

In some embodiments, the combination of non-Clostridium clusters IV and XIVa bacteria (e.g., non-Clostridia class bacteria) strains is selected to mimic the healthy microbiota of an allergen tolerant subject. In some embodiments, the combination of non-Clostridium clusters IV and XIVa bacteria (e.g., non-Clostridia class bacteria) strains is selected to generate a healthy microbiota of an allergen tolerant subject. In some embodiments, the combination of non-Clostridium clusters IV and XIVa bacteria (e.g., non-Clostridia class bacteria) strains is selected to generate the healthy metabolite makeup of an allergen tolerant subject.

In some embodiments, methods are provided herein for (1) preventing development of an allergen hypersensitivity in a subject, and/or (2) desensitizing a subject to an allergen to which the subject has an existing hypersensitivity. In some embodiments, a subject suffers from, or is at risk of suffering from (e.g., based on genetic factors, environmental factors, testing, etc.) a food allergy. In some embodiments, a food allergy is selected from milk, milk proteins, eggs, fish, nuts from trees (e.g., hazelnuts, walnuts, almonds, Brazil nuts, etc.), peanuts (groundnuts), shellfish (e.g., shrimps, mussels, crab, etc.), soy, wheat, etc.). In some embodiments, a subject is treated by the methods herein to prevent development of hypersensitivity to one or the aforementioned allergens. In some embodiments, a composition comprising non-Clostridium clusters IV and XIVa bacteria (e.g., non-Clostridia class bacteria) (e.g., Acidaminococcus intestini, Alistipes putredinis, Bacteroides massiliensis, Bacteroides stercoris, Bifidobacterium adolescentis, Megamonas funiformis, Megamonas hypermegale, Megamonas rupellensis, and taxonomically-related bacteria that similarly support allergen tolerance) is administered to a subject or patient in a pharmaceutically effective amount.

The dosage amount and frequency are selected to create an effective level of non-Clostridium clusters IV and XIVa bacteria (e.g., non-Clostridia class bacteria) without substantially harmful effects. When administered (e.g., orally, rectally, etc.), the dosage will generally comprise at least 1×10⁴ CFU per dose or per day (e.g., 1×10⁴ CFU, 2×10⁴ CFU, 5×10⁴ CFU, 1×10⁵ CFU, 2×10⁵ CFU, 5×10⁵ CFU, 1×10⁶ CFU, 2×10⁶ CFU, 5×10⁶ CFU, 1×10⁷ CFU, 2×10⁷ CFU, 5×10⁷ CFU, 1×10⁸ CFU, 2×10⁸ CFU, 5×10⁸ CFU, 1×10⁹ CFU, 2×10⁹ CFU, 5×10⁹ CFU, 1×10¹⁹ CFU, 2×10¹⁹ CFU, 5×10¹⁹ CFU, 1×10¹¹ CFU, 2×10¹¹ CFU, 5×10¹¹ CFU, 1×10¹² CFU, 2×10¹² CFU, 5×10¹² CFU per dose or per day, or more per dose or per day, including ranges there between) of non-Clostridium clusters IV and XIVa bacteria (e.g., non-Clostridia class bacteria) (e.g., total non-Clostridium clusters IV and XIVa bacteria (e.g., non-Clostridia class bacteria), amount of a particular classification (e.g., strain, species, genus, family, class, etc.) of non-Clostridium clusters IV and XIVa bacteria (e.g., non-Clostridia class bacteria), etc.).

Methods of administering a composition comprising non-Clostridium clusters IV and XIVa bacteria (e.g., non-Clostridia class bacteria) (e.g., an effective level of non-Clostridia class bacteria) include, without limitation, administration in oral, intranasal, topical, sublingual, rectal, and vaginal forms.

In some embodiments, a single dose of a composition comprising non-Clostridium clusters IV and XIVa bacteria (e.g., non-Clostridia class bacteria) (e.g., Acidaminococcus intestini, Alistipes putredinis, Bacteroides massiliensis, Bacteroides stercoris, Bifidobacterium adolescentis, Megamonas funiformis, Megamonas hypermegale, Megamonas rupellensis, and taxonomically-related bacteria that similarly support allergen tolerance) is administered to a subject. In other embodiments, multiple doses are administered over two or more time points, separated by hours, days, weeks, etc. In some embodiments, a composition comprising non-Clostridium clusters IV and XIVa bacteria (e.g., non-Clostridia class bacteria) (e.g., an effective level of non-Clostridium clusters IV and XIVa bacteria (e.g., non-Clostridia class bacteria)) is administered over a long period of time (e.g., chronically), for example, for a period of months or years (e.g., 1, 2, 3, 4, 5, 6, 7, 8, 9, 10, 11, 12, or more months or years; for the subject's lifetime). In such embodiments, a composition comprising non-Clostridium clusters IV and XIVa bacteria (e.g., non-Clostridia class bacteria) (e.g., an effective level of non-Clostridium clusters IV and XIVa bacteria (e.g., non-Clostridia class bacteria)) may be taken on a regular scheduled basis (e.g., daily, weekly, etc.) for the duration of the extended period.

The technology also relates to methods of treating a subject with a composition comprising non non-Clostridium clusters IV and XIVa bacteria (e.g., non-Clostridia class bacteria) (e.g., an effective level of non-Clostridium clusters IV and XIVa bacteria (e.g., non-Clostridia class bacteria)). In some embodiments, the subject has a hypersensitivity to an allergen (e.g., milk and the composition is administered to desensitize the subject). In some embodiments, the subject does not have a sensitivity (e.g., hypersensitivity) to an allergen (e.g., milk) and the composition is administered to prevent development of a sensitivity (e.g., hypersensitivity) or reduce the risk of the subject developing a sensitivity (e.g., hypersensitivity) to the allergen (e.g., milk).

According some embodiments of the technology, a method is provided for treating a subject in need of such treatment with a composition comprising non-Clostridium clusters IV and XIVa bacteria (e.g., non-Clostridia class bacteria) (e.g., Acidaminococcus intestini, Alistipes putredinis, Bacteroides massiliensis, Bacteroides stercoris, Bifidobacterium adolescentis, Megamonas funiformis, Megamonas hypermegale, Megamonas rupellensis, and taxonomically-related bacteria that similarly support allergen tolerance). The method involves administering to the subject a composition comprising non-Clostridium clusters IV and XIVa bacteria (e.g., non-Clostridia class bacteria) (e.g., an effective level of non-Clostridium clusters IV and XIVa bacteria (e.g., non-Clostridia class bacteria)) in any one of the pharmaceutical preparations described above, detailed herein, and/or set forth in the claims. The subject can be any subject in need of such treatment. It should be understood, however, that the composition comprising non-Clostridium clusters IV and XIVa bacteria (e.g., non-Clostridia class bacteria) (e.g., an effective level of non-Clostridium clusters IV and XIVa bacteria (e.g., non-Clostridia class bacteria)) is a member of a class of compositions and the technology is intended to embrace pharmaceutical preparations, methods, and kits containing related derivatives within this class. Another aspect of the technology then embraces the foregoing summary but read in each aspect as if any such derivative is substituted wherever “composition” appears.

In some embodiments, methods and compositions herein find use in the treatment of allergen hypersensitivity (e.g., treatment of a subject that suffers from one or more allergies (e.g., food allergies)). In some embodiments, the compositions herein are administered to a subject suffering from an allergen hypersensitivity to desensitize the subject and/or to reduce the degree of sensitivity of the subject to the allergen. In some embodiments, the compositions described herein (e.g., comprising non-Clostridium clusters IV and XIVa bacteria (e.g., non-Clostridia class bacteria)) are co-administered with one or more additional treatments or therapies. In some embodiments, the additional treatment is also aimed and reducing a subject's sensitivity to the allergen. In some embodiments, compositions herein (e.g., comprising Acidaminococcus intestini, Alistipes putredinis, Bacteroides massiliensis, Bacteroides stercoris, Bifidobacterium adolescentis, Megamonas funiformis, Megamonas hypermegale, Megamonas rupellensis, and taxonomically-related bacteria that similarly support allergen tolerance) are co-administered with allergen immunotherapy. In some embodiments, allergen immunotherapy comprises exposing the subject to initially small amounts of allergen, and increasing the amount of the allergen over time.

In some embodiments, provided herein are compositions and methods for research, screening, and diagnostic applications. For example, in some embodiments, diagnostic applications provide a risk or a measure of gut health. In some embodiments, the level, presence or absence of one or more bacterial members of the microflora (e.g., non-Clostridia class bacteria, Clostridia class bacteria, etc.), is used to provide a diagnosis or prognosis. For example, in some embodiments a lack of or decreased level of one or more bacteria is associated with an increased risk of development of sensitivity (e.g., hypersensitivity) to one or more allergens.

In some embodiments, subjects are tested. Exemplary diagnostic methods are described herein. In some embodiments, intact bacteria are detected (e.g., by detecting surface polypeptides or markers). In other embodiments, bacteria are lysed and nucleic acids or proteins (e.g., corresponding to genes specific to the species of bacteria) are detected.

In some embodiments, bacteria are identified using detection reagents (e.g., a probe, a microarray, e.g., an amplification primer) that specifically interact with a nucleic acid that identifies a particular species of bacteria (e.g., non-Clostridia species, Clostridia species, etc.).

Some embodiments comprise use of nucleic acid sequencing to detect, quantify, and/or identify gut microbiota. The term “sequencing,” as used herein, refers to a method by which the identity of at least 10 consecutive nucleotides (e.g., the identity of at least 20, at least 50, at least 100, or at least 200 or more consecutive nucleotides) of a polynucleotide are obtained. The term “next-generation sequencing” refers to the so-called parallelized sequencing-by-synthesis or sequencing-by-ligation platforms currently employed by Illumina, Life Technologies, and Roche, etc. Next-generation sequencing methods may also include nanopore sequencing methods or electronic-detection based methods such as Ion Torrent technology commercialized by Life Technologies.

Some embodiments of the technology comprise acquiring a gut microbiota sample from a subject. As used herein, “gut microbiota sample” refers to a biological sample comprising a plurality of heterogeneous nucleic acids produced by a subject's gut microbiota. Fecal samples are commonly used in the art to sample gut microbiota. Methods for obtaining a fecal sample from a subject are known in the art and include, but are not limited to, rectal swab and stool collection. Suitable fecal samples may be freshly obtained or may have been stored under appropriate temperatures and conditions known in the art. Methods for extracting nucleic acids from a fecal sample are also well known in the art. The extracted nucleic acids may or may not be amplified prior to being used as an input for profiling the relative abundances of bacterial taxa, depending upon the type and sensitivity of the downstream method. When amplification is desired, nucleic acids may be amplified via polymerase chain reaction (PCR). Methods for performing PCR are well known in the art. Selection of nucleic acids or regions of nucleic acids to amplify are discussed above. The nucleic acids comprising the nucleic acid sample may also be fluorescently or chemically labeled, fragmented, or otherwise modified prior to sequencing or hybridization to an array as is routinely performed in the art.

In some embodiments, nucleic acids are amplified using primers that are compatible with use in, e.g., Illumina's reversible terminator method, Roche's pyrosequencing method (454), Life Technologies's sequencing by ligation (the SOLiD platform) or Life Technologies's Ion Torrent platform. Examples of such methods are described in the following references: Margulies et al (Nature 2005 437: 376-80); Ronaghi et al (Analytical Biochemistry 1996 242: 84-9); Shendure et al (Science 2005 309: 1728-32); Imelfort et al (Brief Bioinform. 2009 10:609-18); Fox et al (Methods Mol Biol. 2009; 553:79-108); Appleby et al (Methods Mol Biol. 2009; 513: 19-39) and Morozova et al (Genomics. 2008 92:255-64), which are incorporated by reference for the general descriptions of the methods and the particular steps of the methods, including all starting products, reagents, and final products for each of the steps.

In another embodiment, the isolated microbial DNA may be sequenced using nanopore sequencing (e.g., as described in Soni et al. Clin Chem 2007 53: 1996-2001, or as described by Oxford Nanopore Technologies). Nanopore sequencing technology is disclosed in U.S. Pat. Nos. 5,795,782, 6,015,714, 6,627,067, 7,238,485 and 7,258,838 and U.S. Pat Appln Nos. 2006003171 and 20090029477.

The isolated microbial fragments may be sequenced directly or, in some embodiments, the isolated microbial fragments may be amplified (e.g., by PCR) to produce amplification products that sequenced. In certain embodiments, amplification products may contain sequences that are compatible with use in, e.g., Illumina's reversible terminator method, Roche's pyrosequencing method (454), Life Technologies's sequencing by ligation (the SOLiD platform) or Life Technologies's Ion Torrent platform, as described above.

In certain embodiments, the sample sequenced may comprise a pool of nucleic acids from a plurality of samples, wherein the nucleic acids in the sample have a molecular barcode to indicate their source. In some embodiments the nucleic acids being analyzed may be derived from a single source (e.g., from different sites or a timecourse in a single subject), whereas in other embodiments, the nucleic acid sample may be a pool of nucleic acids extracted from a plurality of different sources (e.g., a pool of nucleic acids from different subjects), where by “plurality” is meant two or more. Molecular barcodes may allow the sequences from different sources to be distinguished after they are analyzed.

In some embodiments, gut microbiota samples are obtained from a subject (e.g., a healthy subject or a not healthy subject (e.g., a patient or a subject in need of treatment according to the technology provided herein) at any suitable interval of time, varying from minutes to hours apart, days to weeks apart, or even weeks to months apart. Gut microbiota samples may be obtained multiple times a day, week, month or year. The duration of sampling can also vary. For example, the duration of sampling may be for about a month, about 6 months, about 1 year, about 2 years, about 3 years, about 4 years, about 5 years, about 6 years, about 7 years, about 8 years, about 9 years, about 10 years, about 11 years, about 12 years, about 13 years, about 14 years, about 15 years, about 16 years, about 17 years, about 18 years, about 19 years, about 20 years, about 30 years, or more.

In some embodiments, a metabolomics screen is performed on a sample from a subject identify, quantify, etc. various metabolite present. In some embodiments, one or more key metabolites are assayed (e.g., butyrate, propionate, etc.). In some embodiments, the components of a composition for the treatment of a subject (e.g., the quantity and identity of the non-Clostridia class bacteria, the quantity and identity of the Clostridia class bacteria, the quantity and identity of non-bacterial components (e.g., metabolic pathway enzymes, metabolites, etc.)) is determined based on the results of testing (e.g., for metabolites, for microbiota, for allergies, combinations thereof, etc.).

In some embodiments, subjects identified as being at increased risk of allergen hypersensitivity, subjects identified as suffering from allergen hypersensitivity, and/or subject having gut microbiota that does not promote allergen tolerance, may be administered compositions described herein.

In some embodiments, a subject is treated with a composition comprising non-Clostridium clusters IV and XIVa bacteria (e.g., non-Clostridia class bacteria) based on the outcome of a test (e.g., metabolomics testing, microbiomic testing, etc.). Accordingly, in some embodiments, a subject is tested and then treated based on the test results. In some embodiments, a subject is treated and then tested to assess the efficacy of the treatment. In some embodiments, a subsequent treatment is adjusted based on a test result, e.g., the dosage amount, dosage schedule, composition administered, etc. is changed. In some embodiments, a patient is tested, treated, and then tested again to monitor the response to therapy and/or to change the therapy. In some embodiments, cycles of testing and treatment may occur without limitation to the pattern of testing and treating (e.g., test/treat, treat/test, test/treat/test, treat/test/treat, test/treat/test/treat, test/treat/test/treat/test, test/treat/test/test/treat/treat/treat/test, test/treat/treat/test/treat/treat, etc.), the periodicity, or the duration of the interval between each testing and treatment phase.

Although some embodiments herein are described in connection with the treatment or prevention of food allergies, embodiments herein are no limited to as much. Some embodiments herein include the treatment of humans of any suitable age (e.g., infant, child adolescent, adult, etc.) with existing allergies (e.g., food allergies, environmental allergies) or other atopy diseases (e.g., allergic rhinitis, eczema, etc.), or preventing the development of such conditions in a subject (e.g., a subject at risk of developing such a condition).

### Experimental

Experiments were conducted during development of embodiments herein to examine the genotypic and metabolic pathways that were influenced by extensively hydrolyzed casein formula (EHCF) treatment. The metagenome was shotgun sequenced from four samples prior to EHCF-only treatment, and four samples post EHCF-only treatment. Eight pre- and eight post-EHCF+LGG treatment samples were also analyzed; These sixteen metagenomes represent the same 8 children. Four of these children developed tolerance post-treatment with EHCF and the Lactobacillus probiotic, and the other four did not. Analysis of the differences between these groups indicates a formulation of organisms that induces tolerance, based on a rational metabolic pathway design.

### Materials and Methods

### Patient Recruitment, Sampling and Metagenome Sequencing

Fecal samples from Ig-E mediated CMA infants recruited in this study were referred to a tertiary pediatric allergy center (Pediatric Food Allergy Unit at the Department of Translational Medical Science of the University of Naples ‘Federico II’). DNA was isolated from 100-300 mg of fecal material using bead beating before extraction with QIAamp DNA stool mini kit. A total of 25 samples were selected for shotgun metagenome sequencing. Using Illumina's TruSeq library preparation protocol, individual libraries were sequenced on the Illumina Hiseq 2000 platform (100 bp paired end reads with average insert size=180 bp).

### Quality Control and Microbial Community Profiling

Paired end reads were quality-trimmed using nesoni pipeline (github.com/Victorian-Bioinformatics-Consortium/nesoni), with the parameters set at: minimum length=75, quality cutoff=30, adapter trimming yes and ambiguous bases=0. Taxonomical and functional status was assigned to the individual metagenome reads using MetaPhlan2 and Humann2 pipelines, respectively. Biopieces (biopieces.org) package was used to create custom databases for protein coding genes majorly involved in microbial butyrate production (e.g., acetyl-CoA, glutarate, and lysine pathways (Vital et al., 2014; incorporated by reference in its entirety)), using complete microbial genome (bacteria and archaea) sequences downloaded from NCBI (accessed on 2 Jun. 2016). ShortBread (https://bitbucket.org/biobakery/shortbred/wiki/Home) was used to create biomarkers for the butyrate producing genes using Uniprot as reference database (uniprot.org). Metagenomes datasets were mapped against these biomarkers using short_bread_quantify.py script implemented in the ShortBread software. Normalized count of marker genes, expressed in units of RPKMs (reads per kilobase of reference sequence per million sample reads) was further used for pairwise comparisons.

### Metagenome Assembly, Genome Recovery, Curation and Annotation

Quality trimmed metagenome reads were assembled into contigs using IDBA_UD (Peng et al., 2012; incorporated by reference in its entirety) using k-mer length ranging 41 and 61. Metagenome contigs with length <300 bp were excluded from further analysis. Metagenome contigs were assigned to various taxonomical levels using NBC classifier (Rosen et al., 2008; incorporated by reference in its entirety). AGS (average genome size) was computed for each metagenome sample and using MicrobeCensus (Nayfach and Pollard, 2015; incorporated by reference in its entirety). MetaBAT pipeline (Kang et al., 2015; incorporated by reference in its entirety) was used (mode=−specific) for binning genomes from individual metagenome assemblies (contigs >1 kb). Percentage completeness estimations of the reconstructed genomes were performed using CheckM (Parks et al., 2015; incorporated by reference in its entirety). Genomes (n=46) greater than 80% completion were used for detailed downstream analyses. Draft genomes were annotated (Pathway and Enzyme level) using Prokka pipeline (Seemann, 2014; incorporated by reference in its entirety).

### Evaluation and Comparison of the Influence of Physical Environment Across Tolerant and Intolerant

Post-EHCF-LGG samples (n=8) were divided into two groups; tolerant (n=4) and allergic (n=4). In order to evaluate the strength of natural selection, pairwise orthologous protein coding genes were predicted across Post-EHCF-LGG samples using RSD software (Wall et al., 2003; incorporated by reference in its entirety). Genes with less than 80% global alignment cutoff were excluded from the downstream analysis. Pairwise selected orthologous protein coding genes were aligned using ClustalW (Larkin et al., 2007; incorporated by reference in its entirety). Multiple codon alignments were constructed from the corresponding aligned protein sequences using pal2nal script (Suyama et al., 2006; incorporated by reference in its entirety). Final alignments (stop codons removed) were processed for sdN/dS analysis using PAML (Yang, 2007; incorporated by reference in its entirety). To further validate the influence of in situ functional constraints on the observed natural selection patterns the orthologous gene pairs were processed using codon bias variation. Codon deviation coefficient was used as the measure of codon bias across orthologous gene pairs predicted across free-living, endosymbiont and core genotype (Zhang et al., 2012; incorporated by reference in its entirety). Mean value of two orthologous genes was used for the correlation analysis against dN/dS values.

### Results

### Metagenomic Sequencing and Genome Assembly Statistics

In total 1,136,408,125 sequences were generated from 24 samples. The total number of sequences per sample ranged from 45,456,325 to 56,244,528. Metagenomic assembly was performed and sequences were clustered into species-specific genome bins. In total 44 near-complete genotypes were generated.

### Microbial Community Structure was Influenced by Treatment

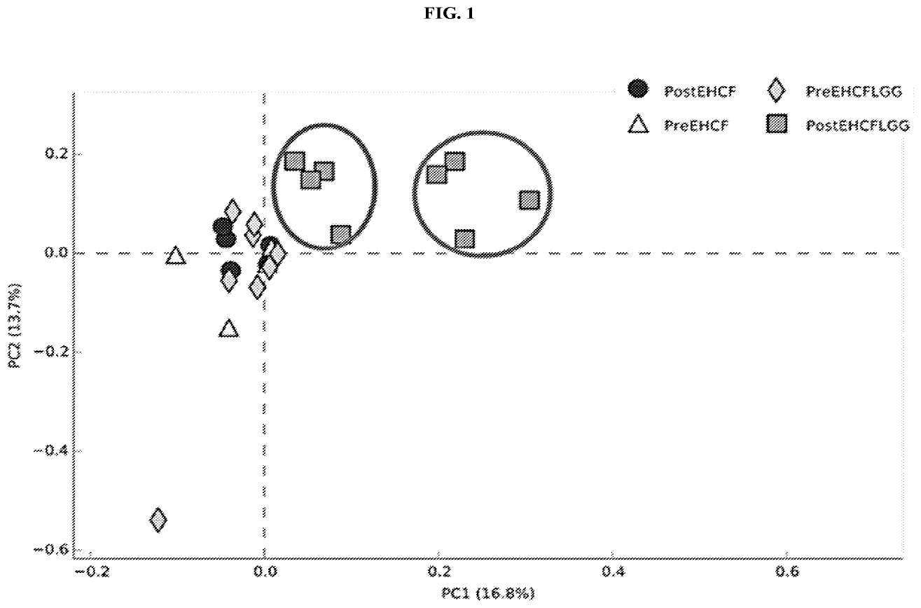

Beta-diversity was calculated based on weighted unifrac distance and the variance in community dissimilarity was visualized using a Principle Component Analysis (PCA; FIG. 1). This demonstrated that apart from an outlier in the pre-EHCFLGG group (which was dominated by Bacteroides), all pre-treatment samples, and the samples from infants post-EHCF-only treatment, clustered together, suggesting similar microbial community composition. Post EHCF+LGG treatment the four infants that became tolerant (postEHCFLGG+) cluster distantly from infants who took EHCF+LGG but didn't develop tolerance (postEHCFLGG−). The postEHCFLGG+samples were significantly different from both the postEHCFLGG- and all remaining samples.

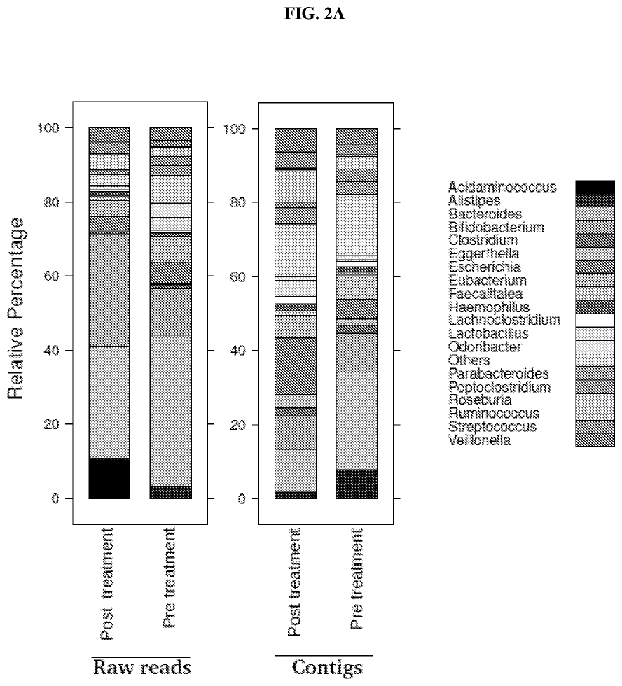

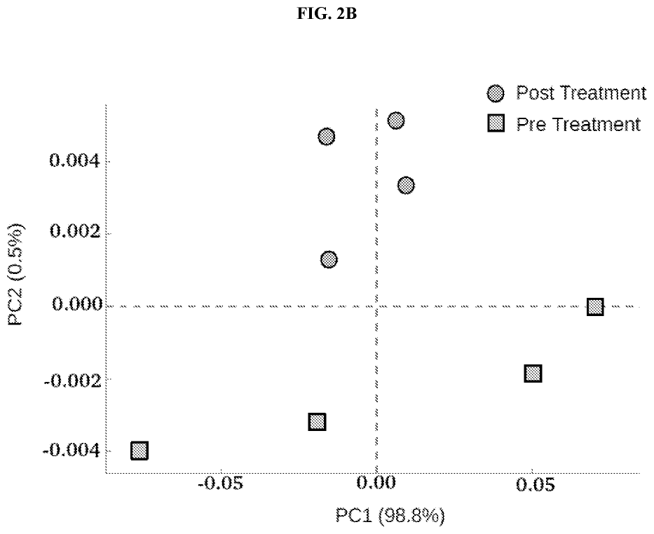

For the EHCFLGG groups, the post-treatment group was more phylogenetically diverse than the pre-treatment group (paired t-test with Bonferroni correction, P<0.005). Bifidobacterium (Post-treatment=raw; 30.393±29.456, contigs=9.165±10.596 and Pre-treatment=raw; 12.556±12.348, contigs; 10.596±2.850) and Bacteroides (Post-treatment=raw; 30.164±34.459, contigs; 11.485±26.304 and Pre-treatment=raw; 40.889±20.335, contigs; 26.304±2.497) were predominant in both groups (FIGS. 2A-B). Roseburia and Eubacterium were more abundant in post-treatment samples than pre-treatment samples (FIG. 1A). Acidaminococcus was significantly more abundant in the post-treatment group (10.390±17.956) compared to the pre-treatment group (0.0232±0.001; FIG. 1A). However, 92% of the Acidaminococcus contigs identified in the post-treatment group were from only one participants sample (oRBC19).

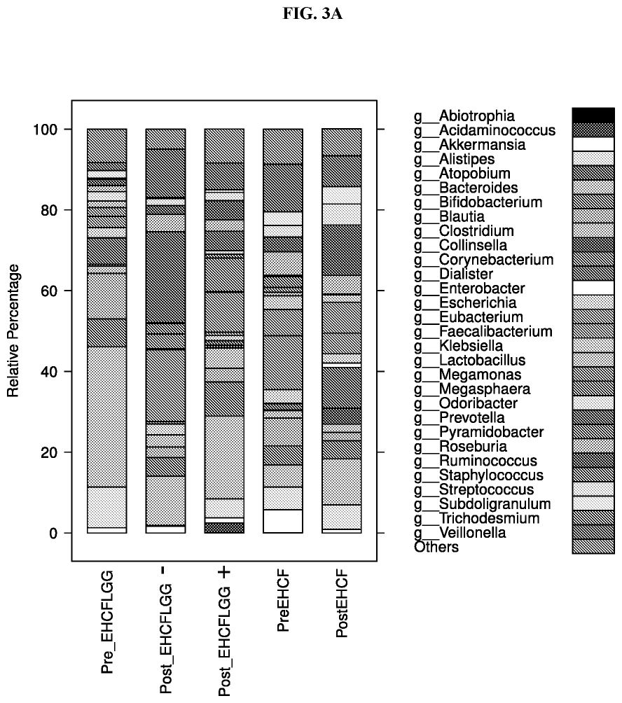

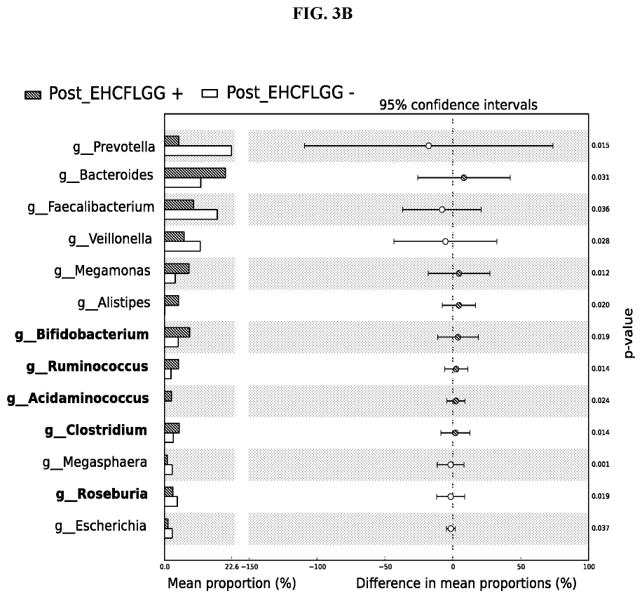

To determine which taxa were significantly differentiating the microbiome of infants that developed tolerance versus those that didn't, genus-level taxonomic analysis was repeated on all samples (FIGS. 3A-B. The microbiome was hardly altered at all in infants that only received EHCF; whereas, there were significant changes between pre- and post-EHCFLGG treatment, including an enrichment of Prevotella, Faecalibacterium, Megamonas, Veillonella, Ruminococcus, Megasphera, etc. Other taxa were reduced in abundance. The significant abundance change between infants that developed tolerance and those that remained sensitive demonstrates a core group of bacteria that became abundant in those that developed tolerance (FIGS. 3A-B). These include, Bacteroides, Megamonas, Alistipes, Bifidobacteria, Ruminococcus, Acidaminococcus and Clostridium. The enrichment of Clostridiales taxa (Ruminococcus, and Clostridium) indicates enrichment of pathways associated with butyrate production. The enrichment of Bifidobacteria, Acidaminococcus, Bacteroides, Megamonas and Alistipes indicates other pathways that are influential in supporting the induction of tolerance.

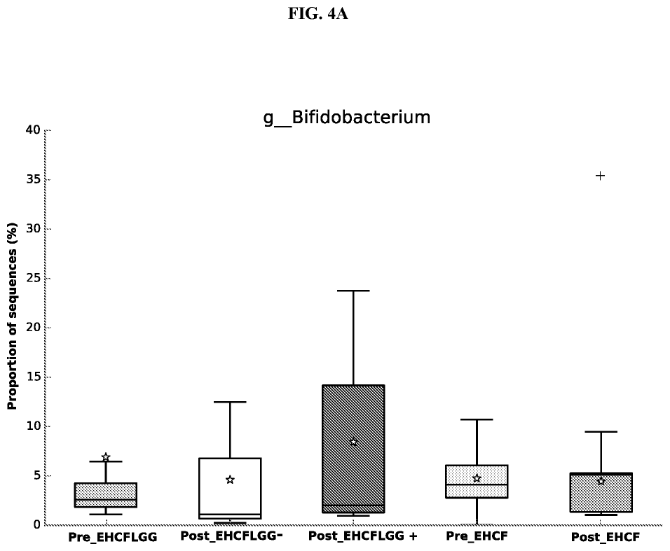

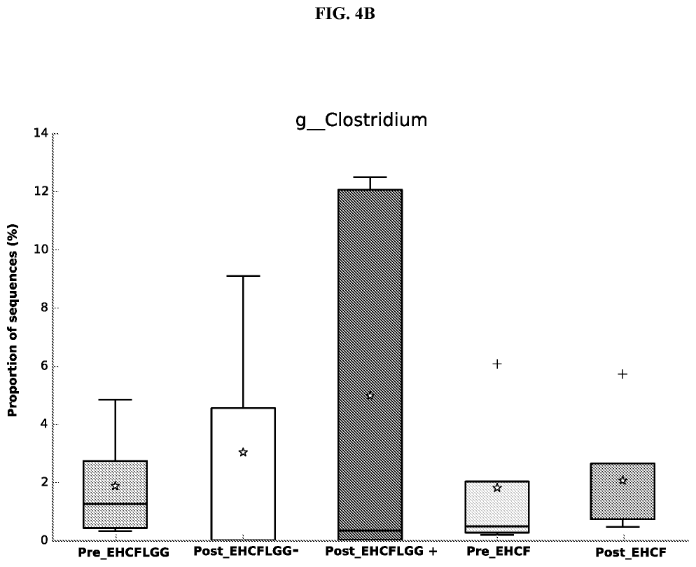

The importance of butyrate producing lineages is associated with a significant increase in the butyrate concentration post treatment for those children who develop tolerance (Berni Canani et al., 2015; incorporated by reference in its entirety). Focusing just on Bifidobacteria and Clostridium, a non-butyrate and a butyrate producing lineage, respectively, show a significant increase in abundance post treatment in those infants who develop tolerance, and that this increase was considerable compared to all other treatment groups (FIGS. 4A-B).

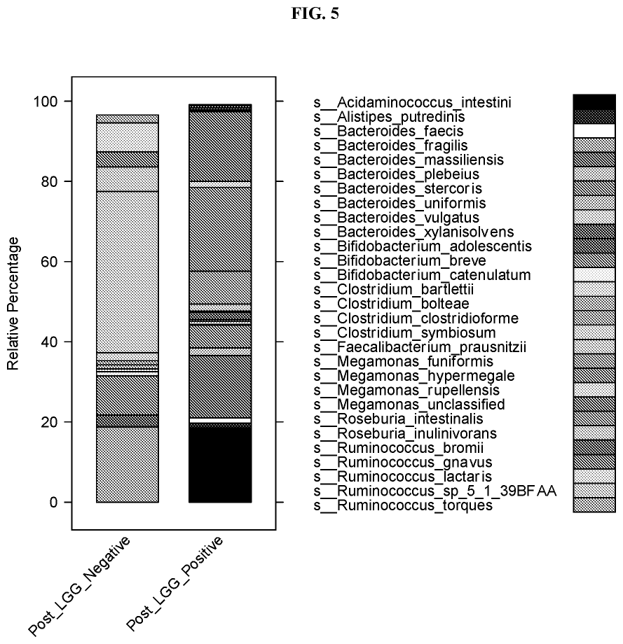

Using genome reconstruction for taxa for which >80% genome re-assembly was achievable, the unique species were identified that were enriched (FIG. 5). These experiments demonstrate that human-isolated taxa including Acidaminococcus intestini, Alistipes putredinis, Bacteroides massiliensis, Bacteroides stercoris, Bifidobacterium adolescentis, Megamonas funiformis, Megamonas hypermegale, Megamonas rupellensis, Megamonas unclassified (new species), Ruminococcus gnavis, etc. support immune activation and Treg recruitment.

### Metabolic Pathway Enrichment

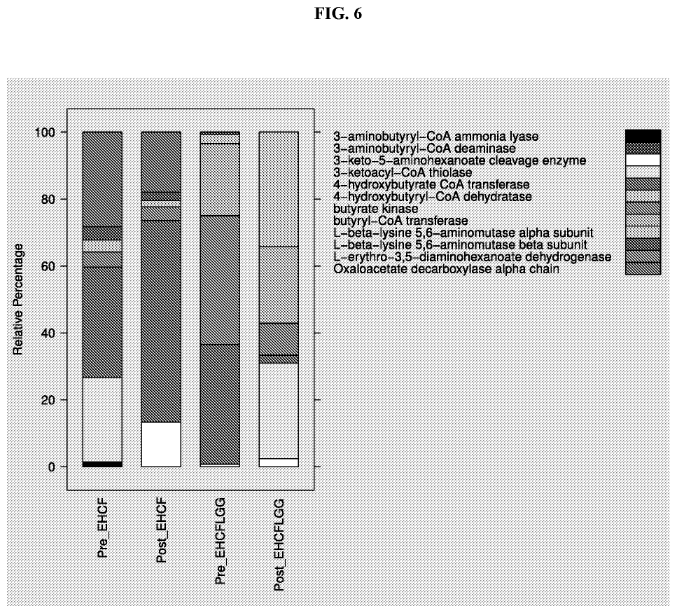

The taxonomic analysis of raw and assembled metagenomic reads suggest that Megamonas, Alistipes, Bacteroides, Bifidobacterium, Ruminococcus, Acidaminococcus and Clostridium might all play a role in supporting the development of tolerance. Analysis of the butyrate synthesis pathway genes that were enriched post-treatment allows for reconstruction of the implications of treatment on these pathways (FIG. 6).

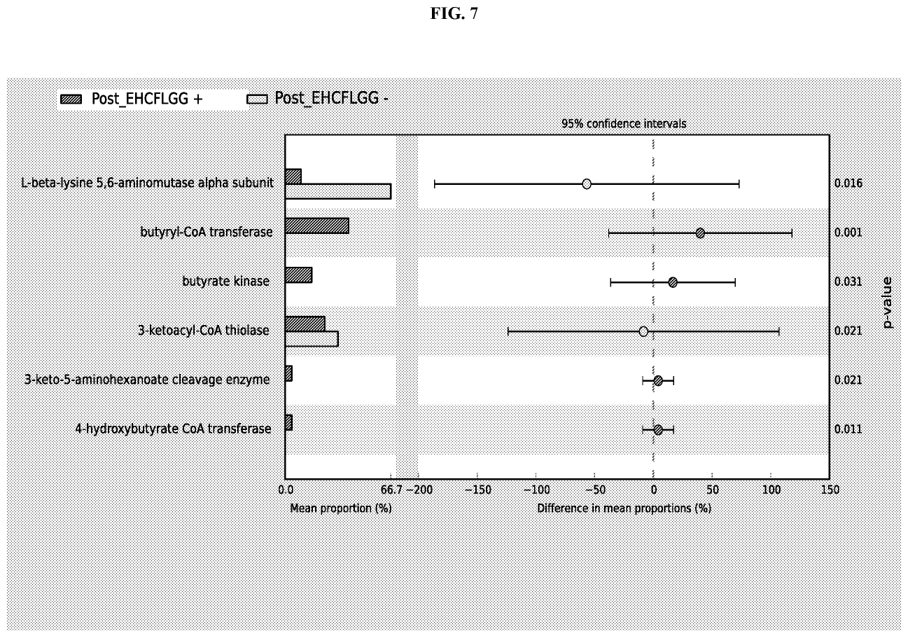

Post treatment with EHCFLGG, infants developed a significant increase in the abundance of 3-ketoacyl-CoA thiolase, which is involved in the production of Acetyl-CoA from 3-ketoacyl-CoA, and therefore provides enrichment in the capacity to perform this transition. This pathway is important in converting Gamma Amino Butyric Acid (GABA) into butyrate. GABA place a significant role in neurological pathway inhibition. Disruption in the balance of GABA in the body leads to consequences for behavioral and gastrointestinal effectors. GABA plays a significant role in controlling the concentration of inflammatory cytokines (e.g., through peripheral macrophages). 3-ketoacyl CoA thiolase is more abundant in infants that do not develop tolerance (FIG. 7), indicating a reduction in GABA concentrations in the gut, and down-regulation of the control of inflammatory cytokines. With control removed, inflammatory cytokines become enriched and exacerbate inflammation, resulting in intolerance.

When infants that became tolerant following treatment are compared with those that did not, multiple other pathways that are differentiated (FIG. 7). For example, the pathways for the production of butyrate from lysine are significantly enriched in infants who did not become tolerant. Enrichment of these pathways leads to a very different environmental context, with side effects including production of ammonia and protons that acidify key environments, contributing to the inflammatory environment. It is contemplated that this explains the abundance of butyrate still present in the stool of the infants who remained intolerant.

Significant enrichment of butyryl-CoA transferase, butyrate kinase and 4-hydroxybutyrate CoA transferase is also observed (FIG. 7), indicating a substantial up-regulation in key metabolic pathways associated with the production of butyrate. It also indicates that in tolerant infants butyrate synthesis is driven by acetyl-CoA, derived from pyruvate and acetate. Butyryl CoA-Acetyl CoA transferase drives the production of Acetyl CoA from acetate and is mediated by the Type XIVa Clostridia (FIG. 8). Meanwhile Megamonas species mediate the production of propionate from pyruvate and this pathway is only upregulated in tolerant infants. Propionate also stimulates Treg accumulation. Finally, reductive acetogenesis is mediated by Marvinbryantia formatexigens, which breaks down formate to produce acetate (as a byproduct of removing hydrogen). This feeds the acetate cycle to produce butyrate.

### Rational Design of a Microbiome-Based Therapeutic for Inflammation Control and Food Allergy Desensitization

Based on comparative metagenome analysis, the taxonomic analysis of raw and assembled metagenomic reads indicates that Megamonas, Alistipes, Bacteroides, Bifidobacterium, Ruminococcus, Acidaminococcus and Clostridium species all play a role in supporting the development of tolerance to cow's milk. Therefore, a consortium was designed around the non-Clostridium clusters IV and XIVa members that were lacking or underrepresented in the dysbiotic gut microbiome of children suffering from cow's milk allergy compared to those children who developed tolerance or were tolerant.

Initial strain selection among the 773 strains currently present in the Virtual Human Microbiome database (vmh.uni.lu/#microbes/search) was made using available information, including an overview of major fermentation products produced by strains isolated from the human gut microbiome. Genome annotation and in silico modeling was subsequently performed to confirm the fermentation products. Specifically, the genome annotation platform, RAST, was used to confirm the presence of key functionalities in the strains providing tolerance to cow's milk. Based on the information around missing and/or underrepresented microorganisms and key functionalities for tolerance, a consortium of 7 strains was designed containing Megamonas funiformis DSM19343, Megamonas hypermegale DSM1672, Acidaminococcus intestini DSM21505, Bacteroides massiliensis DSM17679, Bacteroides stercoris ATCC43183/DSM19555, Alistipes putredinis DSM17216, and Bifidobacterium adolescentis ATCC15703. The strains and their key properties are listed in Table 1. The consortium described in Table 1 provides key functionalities that are lacking or underrepresented in the dysbiotic gut of infants suffering from cow's milk allergy, most notably the synthesis of propionate, a property present in five of the seven strains of which the consortium is comprised. Furthermore, the strains belonging to the family of the Bacteroidaceae also helps with the breakdown of complex biopolymers, including recalcitrant fibers as carbon sources and proteins as a source for amino acids, thus providing key metabolites to other gut microbiome strains, including members of the Clostridium classes IV and XIVa that are underrepresented in the dysbiotic gut microbiome of infants suffering from cow's milk allergy. As such, this seven-strain consortium stimulates the performance of the underrepresented members of the Clostridium classes IV and XIVa.

TABLE 1
Overview of the seven non-Clostridium classes IV and XIVa strains
included in the biotherapeutic for food allergy desensitization (GUT-105A
consortium). Strain selection was based on insights from comparative
metagenomics to complement missing and under-represented strains.

| Strain | | Key fermentation metabolities | | | | | | |
|---|---|---|---|---|---|---|---|---|
| Species | Family | Butyrate | Propionate | Acetate | Lactate | Succinate | Formate | Hydrogen (H₂) |
| Megamonas | Selenomonadaceae | | + | + | + | | | |
| funiformis | | | | | | | | |
| DSM19343 | | | | | | | | |
| Megamonas | Selenomonadaceae | | + | + | | | | |
| hypermegate | | | | | | | | |
| DSM1672 | | | | | | | | |
| Acidaminococcus | Acidaminococcaceae | + | | + | | | | + |
| intestini | | | | | | | | |
| DSM21505 | | | | | | | | |
| Bacteroides | Bacteroidaceae | | + | + | + | + | + | + |
| massilliensis | | | | | | | | |
| DSM17679 | | | | | | | | |
| Bacteroides | Bacteroidaceae | | + | + | + | + | | + |
| stercortis | | | | | | | | |
| ATCC43183/ | | | | | | | | |
| DSM19555 | | | | | | | | |
| Alistipes | Rikenellaceae | | + | + | | + | | + |
| putredinis | | | | | | | | |
| DSM17216 | | | | | | | | |
| Bifidobacterium | Bifidobacteriaceae | | | + | + | + | + | |
| adolescentis | | | | | | | | |
| ATCC15703 | | | | | | | | |

In addition to the seven strains (Table 1), representative for the non-Clostridium classes IV and XIVa bacteria, members of the butyrate-synthesizing Ruminococcaceae (Clostridium class IV) and Lachnospiraceae (Clostridium class XIVa) were also found to be underrepresented in the dysbiotic gut microbiome of children suffering from cow's milk allergy. Propionate and butyrate both stimulate the recruitment and differentiation of Treg cells. Thus, combining the seven strains listed in Table 1 with butyrate producing bacteria results in a synergistic effect for the control of inflammatory symptoms in the gut, as observed in children suffering from food allergy, such as intolerance to cow's milk. Therefore, the butyrate synthesizing bacteria Faecalibacterium prausnitzii DSM17677, Subdoligranulum variabile DSM15176 and Anaerostipes caccae DSM14662 were added to the consortium. The consortium was further optimized by including Marvinbryantia formatexigens DSM14469 to remove the undesirable fermentation products formate and hydrogen, which have an inflammatory effect. Ruminococcus bromii YE202 and Clostridium scindens ATCC35704 were included as Clostridium classes IV and XIVa species that were strongly underrepresented in children suffering from cow's milk allergy. Akkermansia muciniphila ATCC BAA-835 was included for its ability to synthesize propionate. This resulted in the fourteen strains consortium presented in Table 2.

TABLE 2
Overview of the 14 strains included in the biotherapeutic for food
allergy desensitization (GUT-105B consortium). Strain selection was based on
insights from comparative metagenomics to complement missing and under-
represented strains.

| Strain | | Key fermentation metabolities | | | | | | |
|---|---|---|---|---|---|---|---|---|
| Species | Family | Butyrate | Propionate | Acetate | Lactate | Succinate | Formate | Hydrogen (H₂) |
| Megamonas | Selenomonadaceae | | + | + | + | | | |
| funiformis | | | | | | | | |
| DSM19343 | | | | | | | | |
| Megamonas | Selenomonadaceae | | + | + | | | | |
| hypermegate | | | | | | | | |
| DSM1672 | | | | | | | | |
| Acidaminococcus | Acidaminococcaceae | + | | + | | | | + |
| intestini | | | | | | | | |
| DSM21505 | | | | | | | | |
| Bacteroides | Bacteroidaceae | | + | + | + | + | + | + |
| massilliensis | | | | | | | | |
| DSM17679 | | | | | | | | |
| Bacteroides | Bacteroidaceae | | + | + | + | + | | + |
| stercortis | | | | | | | | |
| ATCC43183/ | | | | | | | | |
| DSM19555 | | | | | | | | |
| Alistipes | Rikenellaceae | | + | + | | + | | + |
| putredinis | | | | | | | | |
| DSM17216 | | | | | | | | |
| Bifidobacterium | Bifidobacteriaceae | | | + | + | + | + | |
| adolescentis | | | | | | | | |
| ATCC15703 | | | | | | | | |
| Akkermansia | Akkermansiaceae | | + | + | | | | |
| muciniphila ATCC | | | | | | | | |
| BAA-835 | | | | | | | | |
| Faecalibacterium | Ruminococcaceae | + | | + | + | + | + | |
| prausnitzii | | | | | | | | |
| DSM17677 | | | | | | | | |
| Subdoligranulum | Ruminococcaceae | + | | + | + | + | | |
| variabile | | | | | | | | |
| DSM15176 | | | | | | | | |
| Anaerostipes | Lachnospiraceae | + | | + | + | | | |
| caccae | | | | | | | | |
| DSM14662 | | | | | | | | |
| Marvinbryantia | Lachnospiraceae | | | + | + | | | |
| formatexigens | | | | | | | | |
| DSM14469 | | | | | | | | |
| Clostridium | Lachnospiraceae | | | + | | | | |
| scindens | | | | | | | | |

In some embodiments, compositions and methods are provided utilizing any individual bacteria of combination of the bacteria listed in Tables 1 and 2, exclusively or in combination with other bacteria.

### REFERENCES

The following references, some of which are cited above, are herein incorporated by reference in their entireties.
Berni Canani R, Sangwan N, Stefka A T, Nocerino R, Paparo L, Aitoro R, et al. (2015). Lactobacillus rhamnosus GG-supplemented formula expands butyrate-producing bacterial strains in food allergic infants. ISME J. e-pub ahead of print, doi: 10.1038/ismej.2015.151.
Kang D D, Froula J, Egan R, Wang Z. (2015). MetaBAT, an efficient tool for accurately reconstructing single genomes from complex microbial communities. PeerJ 3: e1165.
Larkin M A, Blackshields G, Brown N P, Chenna R, McGettigan P A, McWilliam H, et al. (2007). Clustal W and Clustal X version 2.0. Bioinformatics 23: 2947-2948.
Nayfach S, Pollard K S. (2015). Average genome size estimation improves comparative metagenomics and sheds light on the functional ecology of the human microbiome. Genome Biol 16: 51.
Parks D H, Imelfort M, Skennerton C T, Hugenholtz P, Tyson G W. (2015). CheckM: assessing the quality of microbial genomes recovered from isolates, single cells, and metagenomes. Genome Res gr.186072.114.
Peng Y, Leung HCM, Yiu S M, Chin FYL. (2012). IDBA-UD: a de novo assembler for single-cell and metagenomic sequencing data with highly uneven depth. Bioinforma Oxf Engl 28: 1420-1428.
Rosen G, Garbarine E, Caseiro D, Polikar R, Sokhansanj B. (2008). Metagenome Fragment Classification Using N-Mer Frequency Profiles. Adv Bioinforma 2008. e-pub ahead of print, doi: 10.1155/2008/205969.
Seemann T. (2014). Prokka: rapid prokaryotic genome annotation. Bioinforma Oxf Engl 30: 2068-2069.
Suyama M, Torrents D, Bork P. (2006). PAL2NAL: robust conversion of protein sequence alignments into the corresponding codon alignments. Nucleic Acids Res 34: W609-612.
Vital M, Howe A C, Tiedje J M. (2014). Revealing the Bacterial Butyrate Synthesis Pathways by Analyzing (Meta)genomic Data. mBio 5: e00889-14.
Wall D P, Fraser H B, Hirsh A E. (2003). Detecting putative orthologs. Bioinformatics 19: 1710-1711.
Yang Z. (2007). PAML 4: Phylogenetic Analysis by Maximum Likelihood. Mol Biol Evol 24: 1586-1591.
Zhang Z, Li J, Cui P, Ding F, Li A, Townsend J P, et al. (2012). Codon Deviation Coefficient: a novel measure for estimating codon usage bias and its statistical significance. BMC Bioinformatics 13: 43.

1. A method of modulating an immune response in a subject, the method comprising administering a composition comprising non-Clostridium clusters IV and XIVa bacteria to the subject.

2. The method of claim 1, wherein the modulation of the immune response comprises preventing allergen hypersensitivity or an inflammatory response.

3. The method of claim 1, wherein the modulation of the immune response comprises desensitizing a subject to an allergen or treating an allergen hypersensitivity or an inflammatory response.

4. The method of claim 2, wherein the subject is at risk of developing allergen hypersensitivity or an inflammatory condition.

5. The method of claim 3, wherein the subject suffers from hypersensitivity to an allergen or a chronic inflammatory condition.

6. The method of claim 5, wherein the subject suffers from hypersensitivity to one or more foods from the group consisting of milk, milk proteins, eggs, fish, shellfish, hazelnuts, walnuts, almonds, Brazil nuts, peanuts, shrimps, mussels, crab, soy, and wheat.

7. The method of one of claims 1-3, wherein the non-Clostridium clusters IV and XIVa bacteria comprises one or more species selected from the phyla Actinobacteria, Bacteroidetes, and Firmicutes.

8. The method of claim 7, wherein the non-Clostridium clusters IV and XIVa bacteria comprises one or more species selected from the phylum Actinobacteria and genus Bifidobacteria.

9. The method of claim 8, wherein the non-Clostridium clusters IV and XIVa bacteria comprises Bifidobacterium adolescentis.

10. The method of claim 7, wherein the non-Clostridium clusters IV and XIVa bacteria comprises one or more species selected from the phylum Bacteroidetes and the class Bacteroidia.

11. The method of claim 10, wherein the non-Clostridium clusters IV and XIVa bacteria comprises one or more species of a genus selected from the group consisting of Rikenella, Alistipes, Anaerocella, Porphyromonas, Prevotella, Hallella, and Alloprevotella.

12. The method of claim 11, wherein the non-Clostridium clusters IV and XIVa bacteria comprises a Bacteroidia species selected from the group consisting of Alistipes putredinis, Bacteroides massiliensis, and Bacteroides stercoris.

13. The method of claim 7, wherein the non-Clostridium clusters IV and XIVa bacteria comprises one or more species selected from the phylum Firmicutes and the class Negativicutes.

14. The method of claim 13, wherein the non-Clostridium clusters IV and XIVa bacteria comprises one or more species of a genus selected from the group consisting of Megamonas, Acidaminococcus, Succinispira, Megasphaera, Dialister, Pelosiunus, and Veillonella.

15. The method of claim 14, wherein the non-Clostridium clusters IV and XIVa bacteria comprises one or more species selected from the group consisting of Acidaminococcus intestini, Megamonas funiformis, Megamonas hypermegale, and Megamonas rupellensis.

16. The method of one or claims 1-3, wherein the non-Clostridium clusters IV and XIVa bacteria comprises bacteria of one or more genera selected from the group consisting of Megamonas, Acidaminococcus, Succinispira, Megasphaera, Dialister, Pelosiunus, Veillonella, Rikenella, Alistipes, Anaerocella, Porphyromonas, Prevotella, Hallella, and Alloprevotella.

17. The method of one or claims 1-3, wherein the non-Clostridium clusters IV and XIVa bacteria comprises one or more species selected from the group consisting of Acidaminococcus intestini, Alistipes putredinis, Bacteroides massiliensis, Bacteroides stercoris, Bifidobacterium adolescentis, Megamonas funiformis, Megamonas hypermegale, Megamonas rupellensis, and taxonomically-related bacteria that similarly support allergen tolerance.

18. The method of one or claims 1-3, wherein administering the composition supporting butyrate production by Clostridia class bacteria in the subject.

19. The method of one or claims 1-3, wherein administering the composition activates regulator T cell accumulation.
The method of one or claims 1-3, wherein administering the composition causes a decrease in the secretion of a pro-inflammatory cytokine or an enhanced secretion of an anti-inflammatory cytokine by a population of human peripheral blood mononuclear cells at levels sufficient to allow for immune response modulation.

20. The method of one or claims 1-3, wherein administering the composition results in increased catabolism of allergens.

21. The method of claim one or claims 1-3, wherein the composition comprises at least 10⁴ colony forming units (CFU) of non-Clostridium clusters IV and XIVa bacteria.

22. The method of one or claims 1-3, wherein the subject has abnormal gut microbiota.

23. The method of one or claims 1-3, wherein the subject is a human.

24. The method of claim 23, wherein the subject is a human infant, neonate, or child.

25. The method of one or claims 1-3, wherein the composition is administered orally.

26. The method of one or claims 1-3, wherein the composition is administered rectally.

27. The method of one or claims 1-3, further comprising assaying the microbiome and/or metabolome of the subject.

28. The method of claim 27, wherein assaying the microbiome comprises testing the presence, absence, or amount of one or more non-Clostridia and/or Clostridia bacteria in the gut of the subject.

29. The method of claim 27, wherein assaying the metabolome comprises quantifying amount of one or more metabolites in the gut of the subject.

30. The method of claim 29, wherein one of said one or more metabolites is butyrate.

31. The method of claim 27, wherein the assaying is performed on the subject before and/or after administration of the composition.

32. The method of one or claims 1-3, wherein the composition is co-administered with one or more additional active agents.

33. The method of claim 32, wherein the additional active agent comprises a probiotic component or a prebiotic component.

34. The method of claim 32, wherein the additional active agent comprises a Clostridia class bacteria.

35. A pharmaceutical composition comprising non-Clostridium clusters IV and XIVa bacteria and a pharmaceutically acceptable carrier.

36. The pharmaceutical composition of claim 35, wherein the non-Clostridium clusters IV and XIVa bacteria comprises one or more species selected from the phyla Actinobacteria, Bacteroidetes, and Firmicutes.

37. The pharmaceutical composition of claim 36, wherein the non-Clostridium clusters IV and XIVa bacteria comprises one or more species selected from the phylum Actinobacteria and genus Bifidobacteria.

38. The pharmaceutical composition of claim 37, wherein the non-Clostridium clusters IV and XIVa bacteria comprises Bifidobacterium adolescentis.

39. The pharmaceutical composition of claim 36, wherein the non-Clostridium clusters IV and XIVa bacteria comprises one or more species selected from the phylum Bacteroidetes and the class Bacteroidia.

40. The pharmaceutical composition of claim 39, wherein the non-Clostridium clusters IV and XIVa bacteria a comprises one or more species of a genus selected from the group consisting of Rikenella, Alistipes, Anaerocella, Porphyromonas, Prevotella, Hallella, and Alloprevotella.

41. The pharmaceutical composition of claim 40, wherein the non-Clostridium clusters IV and XIVa bacteria comprises a Bacteroidia species selected from the group consisting of Alistipes putredinis, Bacteroides massiliensis, and Bacteroides stercoris.

42. The pharmaceutical composition of claim 36, wherein the non-Clostridium clusters IV and XIVa bacteria comprises one or more species selected from the phylum Firmicutes and the class Negativicutes.

43. The pharmaceutical composition of claim 42, wherein the non-Clostridium clusters IV and XIVa bacteria comprises one or more species of a genus selected from the group consisting of Megamonas, Acidaminococcus, Succinispira, Megasphaera, Dialister, Pelosiunus, and Veillonella.

44. The pharmaceutical composition of claim 43, wherein non-Clostridium clusters IV and XIVa bacteria comprises one or more species selected from the group consisting of Acidaminococcus intestini, Megamonas funiformis, Megamonas hypermegale, and Megamonas rupellensis.

45. The pharmaceutical composition of claim 35, wherein the non-Clostridium clusters IV and XIVa bacteria of one or more genera selected from the group consisting of Megamonas, Acidaminococcus, Succinispira, Megasphaera, Dialister, Pelosiunus, Veillonella, Rikenella, Alistipes, Anaerocella, Porphyromonas, Prevotella, Hallella, and Alloprevotella.

46. The pharmaceutical composition of claim 35, wherein the non-Clostridium clusters IV and XIVa bacteria comprises one or more species selected from the group consisting of Acidaminococcus intestini, Alistipes putredinis, Bacteroides massiliensis, Bacteroides stercoris, Bifidobacterium adolescentis, Megamonas funiformis, Megamonas hypermegale, Megamonas rupellensis, and taxonomically-related bacteria that similarly support allergen tolerance.

47. The pharmaceutical composition of claim 35, comprising a therapeutically effective amount of non-Clostridium clusters IV and XIVa bacteria.

48. The pharmaceutical composition of claim 47, wherein a therapeutically effective amount of non-Clostridium clusters IV and XIVa bacteria is an amount sufficient to increase butyrate production by Clostridia class bacteria in the subject.

49. The pharmaceutical composition of claim 47, wherein a therapeutically effective amount of non-Clostridium clusters IV and XIVa bacteria is an amount sufficient to activate regulator T cell accumulation in the subject.
The pharmaceutical composition of claim 47, wherein a therapeutically effective amount of non-Clostridium clusters IV and XIVa bacteria is an amount sufficient to cause a decrease in the secretion of a pro-inflammatory cytokine or an enhanced secretion of an anti-inflammatory cytokine by a population of human peripheral blood mononuclear cells at levels sufficient to allow for immune response modulation.

50. The pharmaceutical composition of claim 47, wherein a therapeutically effective amount of non-Clostridium clusters IV and XIVa bacteria is an amount sufficient to increase catabolism of allergens in the subject.

51. The pharmaceutical composition of claim 47, wherein the composition comprises at least 10⁴ colony forming units (CFU) of non-Clostridium clusters IV and XIVa bacteria.

52. The pharmaceutical composition of claim 35, further comprising a probiotic or a prebiotic.

53. The pharmaceutical composition of claim 35, formulated for administration to a human newborn, neonate, infant, or child.

54. The pharmaceutical composition of claim 35, wherein the bacteria are alive.

55. The pharmaceutical composition of claim 35, wherein the bacteria are sporulated.

56. The pharmaceutical composition of claim 35, formulated for oral administration.

57. The pharmaceutical composition of claim 35, formulated for rectal administration.

58. The pharmaceutical composition of claim 35, wherein the pharmaceutical composition is a nutraceutical or a food.

59. Use of a composition comprising non-Clostridium clusters IV and XIVa bacteria to manufacture a medicament for administration to a subject.

60. Use of a composition comprising non-Clostridium clusters IV and XIVa bacteria to treat or prevent allergen hypersensitivity in a subject.

61. A pharmaceutical composition comprising a bacteria and a pharmaceutically acceptable carrier, wherein the bacteria comprise a biologically pure culture of a strain from species: Megamonas funiformis, Megamonas hypermegale, Acidaminococcus intestine, Bacteroides massiliensis, Bacteroides stercoris, Alistipes putredinis, and Bifidobacterium adolescentis.

62. The pharmaceutical composition of claim 61, wherein the bacteria comprise a biologically pure culture of: Megamonas funiformis DSM19343, Megamonas hypermegale DSM1672, Acidaminococcus intestine DSM21505, Bacteroides massiliensis DSM17679, Bacteroides stercoris ATCC43183/DSM19555, Alistipes putredinis DSM17216, and Bifidobacterium adolescentis ATCC15703.

63. A pharmaceutical composition comprising a bacteria and a pharmaceutically acceptable carrier, wherein the bacteria consists of a biologically pure culture of a strain from species: Megamonas funiformis, Megamonas hypermegale, Acidaminococcus intestine, Bacteroides massiliensis, Bacteroides stercoris, Alistipes putredinis, and Bifidobacterium adolescentis.

64. The pharmaceutical composition of claim 63, wherein the bacteria consist of a biologically pure culture of: Megamonas funiformis DSM19343, Megamonas hypermegale DSM1672, Acidaminococcus intestine DSM21505, Bacteroides massiliensis DSM17679, Bacteroides stercoris ATCC43183/DSM19555, Alistipes putredinis DSM17216, and Bifidobacterium adolescentis ATCC15703.

65. The pharmaceutical composition of claim 61, further comprising species: Faecalibacterium prausnitzii, Subdoligranulum variabile, Anaerostipes caccae, Marvinbryantia formatexigens, Clostridium scindens, and Ruminococcus bromii.

66. The pharmaceutical composition of claim 65, wherein the strain is: Faecalibacterium prausnitzii DSM17677, Subdoligranulum variabile DSM15176, Anaerostipes caccae DSM14662, Marvinbryantia formatexigens DSM14469, Clostridium scindens ATCC35704, and Ruminococcus bromii YE202.

67. The pharmaceutical composition of claim 63, further comprising species: Faecalibacterium prausnitzii, Subdoligranulum variabile, Anaerostipes caccae, Marvinbryantia formatexigens, Clostridium scindens, and Ruminococcus bromii.

68. The pharmaceutical composition of claim 67, wherein the strain is: Faecalibacterium prausnitzii DSM17677, Subdoligranulum variabile DSM15176, Anaerostipes caccae DSM14662, Marvinbryantia formatexigens DSM14469, Clostridium scindens ATCC35704, and Ruminococcus bromii YE202.

69. The pharmaceutical composition of claim 61 or 63, further comprising a biologically pure culture of a strain of species Akkermansia muciniphila or Akkermansia muciniphila ATCC BAA-835.
